(19) United States
(12) Patent Application Publication
(10) Pub. No.: US 2010/0317686 A1
(43) Pub. Date: Dec. 16, 2010

(54) N-HETEROCYCLIC-6-HETEROCYCLIC-IMIDAZO[1,2-a]PYRIDINE-2-CARBOXAMIDE DERIVATIVES, PREPARATION AND THERAPEUTIC USE THEREOF

(75) Inventor: Jean-Francois PEYRONEL, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 12/828,376

(22) Filed: Jul. 1, 2010

Related U.S. Application Data
(63) Continuation of application No. PCT/FR2008/001838, filed on Dec. 31, 2008.

(30) Foreign Application Priority Data
Jan. 2, 2008 (FR) 0800006

(51) Int. Cl. A61K 31/437; C07D 471/04; A61P 29/00; A61P 25/16; A61P 25/28; A61P 37/00; A61P 25/18; A61P 25/24; A61P 25/30; A61P 25/00; A61P 19/10; A61P 25/08; A61P 35/00

(52) U.S. Cl. 514/300; 546/121

(57) ABSTRACT

Compounds of formula (I):

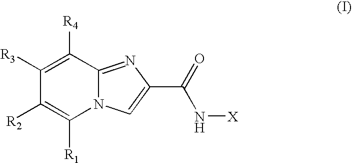

in which:
X, R₁, R₂, R₃ and R₄ are as defined in the disclosure, or an acid addition salt thereof; and therapeutic use thereof.

The present invention relates to imidazo[1,2-a]pyridine-2-carboxamide derivatives, to their preparation and to their therapeutic use in the treatment or prevention of diseases involving the Nurr-1 nuclear receptors, also known as NR4A2, NOT, TINUR, RNR-1 and HZF3.

One subject of the present invention is compounds of formula (I):

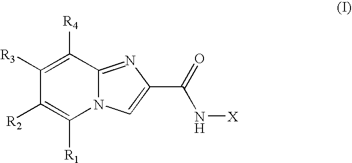

in which:
X represents a heterocyclic group optionally substituted with one or more groups chosen, independently of each other, from the following groups or atoms: halogen, (C₁-C₆)alkoxy, (C₁-C₆)alkyl, NRaRb, cyano, oxido, COOR₈, the alkyl and alkoxy groups possibly being substituted with one or more halogen atoms;
R₁ represents a hydrogen atom, a halogen atom, a group (C₁-C₆)alkoxy, a group (C₁-C₆)alkyl, amino or NRaRb; the alkyl and alkoxy groups possibly being substituted with one or more halogen atoms, a hydroxyl or amino group, or a group (C₁-C₆)alkoxy;
R₂ represents a heterocyclic group, this group possibly being substituted with one or more groups chosen, independently of each other, from the following groups or atoms: hydroxyl, (C₁-C₆)alkyl, (C₁-C₆)alkoxy, halogen, cyano, NRaRb, —CO—R₅, —CO—NR₆R₇, —CO—O—R₈, —NR₉—CO—R₁₀, the groups (C₁-C₆)alkyl and (C₁-C₆)alkoxy being optionally substituted with one or more halogen atoms or hydroxyl, NRaRb or oxido groups;
R₃ represents a hydrogen atom, a group (C₁-C₆)alkyl, a group (C₁-C₆)alkoxy or a halogen atom;
R₄ represents a hydrogen atom, a group (C₁-C₄)alkyl, a group (C₁-C₄)alkoxy or a fluorine atom;
R₅ represents a hydrogen atom, a phenyl group or a group (C₁-C₆)alkyl;
R₆ and R₇, which may be identical or different, represent a hydrogen atom or a group (C₁-C₆)alkyl, or form, with the nitrogen atom that bears them, a 4- to 7-membered ring optionally including another heteroatom chosen from N, O and S;
R₈ represents a group (C₁-C₆)alkyl;
R₉ and R₁₀, which may be identical or different, represent a hydrogen atom or a group (C₁-C₆)alkyl;
Ra and Rb represent, independently of each other, a hydrogen atom, a group (C₁-C₆)alkyl or form, with the nitrogen atom that bears them, a 4- to 7-membered ring optionally including another heteroatom chosen from N, O and S;
   in the form of the base or of an acid-addition salt.

The compounds of formula (I) may comprise one or more asymmetric carbon atoms. They may thus exist in the form of enantiomers or diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or solvates, i.e. in the form of associations or combinations with one or more water molecules or with a solvent. Such hydrates and solvates also form part of the invention.

In the context of the present invention, the following definitions apply:
a halogen atom: a fluorine, a chlorine, a bromine or an iodine;
an alkyl group: a linear, branched or cyclic, saturated aliphatic group, optionally substituted with a linear, branched or cyclic, saturated alkyl group. Examples that may be mentioned include methyl, ethyl, propyl, isopropyl, butyl, isobutyl, tert-butyl, cyclopropyl, cyclobutyl, cyclopentyl, cyclohexyl, methylcyclopropyl, etc. groups;
an alkoxy group: a radical —O-alkyl in which the alkyl group is as defined previously;
a heterocyclic group: a monocyclic or bicyclic group comprising from 5 to 10 atoms including from 1 to 4 heteroatoms chosen from N, O and S; this cyclic group is aromatic, unsaturated or partially unsaturated or oxidized, and is connected via a carbon atom. Examples of heterocyclic groups that may be mentioned include: pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine, triazine, pyrrolopyrrole, pyrroloimidazole, pyrrolopyrazole, pyrrolotriazole, imidazoimidazole, imidazopyrazole, imidazotriazole, indole, isoindole, benzimidazole, indazole, indolizine, benzofuran, isobenzofuran, benzothiophene, benzo[c]thiophene, pyrrolopyridine, imidazopyridine, pyrazolopyridine, triazolopyridine, tetrazolopyridine, pyrrolopyrimidine, imidazopyrimidine, pyrazolopyrimidine, triazolopyrimidine, tetrazolopyrimidine, pyrrolopyrazine, imidazopyrazine, pyrazolopyrazine, triazolopyrazine, tetrazolopyrazine, pyrrolopyridazine, imidazopyridazine, pyrazolopyridazine, triazolopyridazine, tetrazolopyridazine, pyrrolotriazine, imidazotriazine, pyrazolotriazine, triazolotriazine, tetrazolotriazine, furopyridine, furopyrimidine, furopyrazine, furopyridazine, furotriazine, oxazolopyridine, oxazolopyrimidine, oxazolopyrazine, oxazolopyridazine, oxazolotriazine, isoxazolopyridine, isoxazolopyrimidine, isoxazolopyrazine, isoxazolopyridazine, isoxazolotriazine, oxadiazolopyridine, oxadiazolopyrimidine, oxadiazolopyrazine, oxadiazolopyridazine, oxadiazolotriazine, benzoxazole, benzisoxazole, benzoxadiazole, thienopyridine, thienopyrimidine, thienopyrazine, thienopyridazine, thienotriazine, thiazolopyridine, thiazolopyrimidine, thiazolopyrazine, thiazolopyridazine, thiazolotriazine, isothiazolopyridine, isothiazolopyrimidine, isothiazolopyrazine, isothiazolopyridazine, isothiazolotriazine, thiadiazolopyridine, thiadiazolopyrimidine, thiadiazolopyrazine, thiadiazolopyridazine, thiadiazolotriazine, benzothiazole, benzoisothiazole, benzothiadiazole, quinoline, isoquinoline, cinnoline, phthalazine, quinoxaline, quinazoline, naphthyridine, benzotriazine, pyridopyrimidine, pyridopyrazine, pyridopyridazine, pyridotriazine, pyrimidopyrimidine, pyrimidopyrazine, pyrimidopyridazine, pyrimidotriazine, pyrazinopyrazine, pyrazinopyridazine, pyrazinotriazine, pyridazinopyridazine, pyridazinotriazine.

The heterocyclic group used in the compounds according to the present invention is, for example, a monocyclic heterocyclic group.

Various subgroups of compounds, also forming part of the invention, are defined hereinbelow.

Among the compounds of formula (I) that are subjects of the invention, a first group of compounds is constituted of compounds for which:
X represents a heterocyclic group optionally substituted with one or more groups chosen, independently of each other, from halogen atoms;

the other substituents being as defined previously.

Among the compounds of formula (I) that are subjects of the invention, a second group of compounds is constituted of compounds for which:

X represents a pyridine, isoxazole, thiazole, thiadiazole, pyrazole, thiophene or oxazole group, these groups being optionally substituted with a halogen atom;

the other substituents being as defined previously.

Among the compounds of formula (I) that are subjects of the invention, a third group of compounds is constituted of compounds for which:

R₁, R₃ and R₄ represent a hydrogen atom;

the other substituents being as defined previously.

Among the compounds of formula (I) that are subjects of the invention, a fourth group of compounds is constituted of compounds for which:

R₂ represents a heterocyclic group, this group possibly being substituted with one or more groups chosen, independently of each other, from the following groups or atoms: (C₁-C₆)alkyl, NRaRb, the group(s) (C₁-C₆)alkyl being optionally substituted with one or more halogen atoms or hydroxyl groups,

Ra and Rb represent, independently of each other, a hydrogen atom or a group (C₁-C₆)alkyl;

the other substituents being as defined previously.

Among the compounds of formula (I) that are subjects of the invention, a fifth group of compounds is constituted of compounds for which:

R₂ represents a pyridine, thiophene, imidazole, pyrrole, furan, oxazole, triazole or pyrazole group, these groups being optionally substituted with an NH₂ or hydroxymethyl group;

the other substituents being as defined previously.

Among the compounds of formula (I) that are subjects of the invention, a sixth group of compounds is constituted of compounds for which:

X represents a pyridine, isoxazole, thiazole, thiadiazole, pyrazole, thiophene or oxazole group, these groups being optionally substituted with a fluorine atom,

R₂ represents a pyridine, thiophene, imidazole, pyrrole, furan, oxazole, triazole or pyrazole group, these groups being optionally substituted with an NH₂ or hydroxymethyl group,

R₁, R₃ and R₄ represent a hydrogen atom.

Among the compounds of formula (I) that are subjects of the invention, a seventh group of compounds is constituted of compounds for which:

X represents a pyridine, isoxazole, thiazole, thiadiazole, pyrazole or thiophene group, these groups being optionally substituted with a fluorine atom;

R₂ represents a pyridine, thiophene, imidazole, pyrrole, furan, oxazole or triazole group, these groups being optionally substituted with an NH₂ or hydroxymethyl group;

R₁, R₃ and R₄ represent a hydrogen atom, in the form of the base or of an acid-addition salt.

Among the compounds of formula (I) that are subjects of the invention, mention may be made especially of the following compounds:
6-(6-Aminopyridin-2-yl)-N-(pyridin-2-yl)imidazo[1,2-a]pyridine-2-carboxamide and its hydrochloride (1:2)
6-(1H-Imidazol-4-yl)-N-(pyridin-2-yl)imidazo[1,2-a]pyridine-2-carboxamide
N,6-Di(pyridin-2-yl)imidazo[1,2-a]pyridine-2-carboxamide
N-(Pyridin-2-yl)-6-(thiophen-3-yl)imidazo[1,2-a]pyridine-2-carboxamide
N-(Isoxazol-4-yl)-6-(pyridin-2-yl)imidazo[1,2-a]pyridine-2-carboxamide
N-(6-Fluoropyridin-2-yl)-6-(pyridin-2-yl)imidazo[1,2-a]pyridine-2-carboxamide
6-(Pyridin-2-yl-N-(thiazol-2-yl)imidazo[1,2-a]pyridine-2-carboxamide
N-(Isoxazol-3-yl)-6-(pyridin-2-yl)imidazo[1,2-a]pyridine-2-carboxamide
6-(Pyridin-2-yl-N-(1,3,4-thiadiazol-2-yl)imidazo[1,2-a]pyridine-2-carboxamide
N-(1H-Pyrazol-3-yl)-6-(pyridin-2-yl)imidazo[1,2-a]pyridine-2-carboxamide
6-(Pyridin-2-yl-N-(thiophen-3-yl)imidazo[1,2-a]pyridine-2-carboxamide
6-(1H-Pyrrol-3-yl)-N-(thiazol-2-yl)imidazo[1,2-a]pyridine-2-carboxamide
N-(Isoxazol-4-yl)-6-(1H-pyrazol-3-yl)imidazo[1,2-a]pyridine-2-carboxamide
6-(Furan-2-yl)-N-(thiazol-2-yl)imidazo[1,2-a]pyridine-2-carboxamide
6-(Furan-2-yl)-N-(isoxazol-4-yl)imidazo[1,2-a]pyridine-2-carboxamide
6-(Oxazol-2-yl)-N-(thiazol-2-yl)imidazo[1,2-a]pyridine-2-carboxamide
N-(Isoxazol-3-yl)-6-(oxazol-2-yl)imidazo[1,2-a]pyridine-2-carboxamide
N-(Isoxazol-4-yl)-6-(oxazol-2-yl)imidazo[1,2-a]pyridine-2-carboxamide
6-(Furan-3-yl)-N-(thiazol-2-yl)imidazo[1,2-a]pyridine-2-carboxamide
6-(Furan-3-yl)-N-(isoxazol-4-yl)imidazo[1,2-a]pyridine-2-carboxamide
6-[5-(Hydroxymethyl)furan-2-yl]-N-(thiazol-2-yl)imidazo[1,2-a]pyridine-2-carboxamide
6-[5-(Hydroxymethyl)furan-2-yl]-N-(isoxazol-3-yl)imidazo[1,2-a]pyridine-2-carboxamide
6-[5-(Hydroxymethyl)furan-2-yl]-N-(isoxazol-4-yl)imidazo[1,2-a]pyridine-2-carboxamide
6-(1H-Imidazol-4-yl)-N-(isoxazol-3-yl)imidazo[1,2-a]pyridine-2-carboxamide
6-(6-Aminopyridin-2-yl)-N-(thiazol-2-yl)imidazo[1,2-a]pyridine-2-carboxamide
6-(1H-Pyrrol-3-yl)-N-(thiophen-3-yl)imidazo[1,2-a]pyridine-2-carboxamide
6-(Furan-2-yl)-N-(pyridin-2-yl)imidazo[1,2-a]pyridine-2-carboxamide
N-(6-Fluoropyridin-2-yl)-6-(furan-2-yl)imidazo[1,2-a]pyridine-2-carboxamide
6-(Furan-2-yl)-N-(thiophen-3-yl)imidazo[1,2-a]pyridine-2-carboxamide
6-(Furan-2-yl)-N-(isoxazol-3-yl)imidazo[1,2-a]pyridine-2-carboxamide
6-(Furan-2-yl)-N-(1,3,4-thiadiazol-2-yl)imidazo[1,2-a]pyridine-2-carboxamide
6-(Oxazol-2-yl)-N-(pyridin-2-yl)imidazo[1,2-a]pyridine-2-carboxamide
N-(6-Fluoropyridin-2-yl)-6-(oxazol-2-yl)imidazo[1,2-a]pyridine-2-carboxamide
6-(Oxazol-2-yl)-N-(thiophen-3-yl)imidazo[1,2-a]pyridine-2-carboxamide
6-(Furan-3-yl)-N-(pyridin-2-yl)imidazo[1,2-a]pyridine-2-carboxamide
N-(6-Fluoropyridin-2-yl)-6-(furan-3-yl)imidazo[1,2-a]pyridine-2-carboxamide
6-(Furan-3-yl)-N-(thiophen-3-yl)imidazo[1,2-a]pyridine-2-carboxamide
6-(Furan-3-yl)-N-(isoxazol-3-yl)imidazo[1,2-a]pyridine-2-carboxamide
6-(Furan-3-yl)-N-(1,3,4-thiadiazol-2-yl)imidazo[1,2-a]pyridine-2-carboxamide
6-[5-(Hydroxymethyl)furan-2-yl]-N-(pyridin-2-yl)imidazo[1,2-a]pyridine-2-carboxamide
6-[5-(Hydroxymethyl)furan-2-yl]-N-(thiophen-3-yl)imidazo[1,2-a]pyridine-2-carboxamide
6-(Furan-2-yl)-N-(1H-pyrazol-3-yl)imidazo[1,2-a]pyridine-2-carboxamide
N-(Thiophen-3-yl)-6-(1H-1,2,4-triazol-3-yl)imidazo[1,2-a]pyridine-2-carboxamide
6-(6-Aminopyridin-2-yl)-N-(6-fluoropyridin-2-yl)imidazo[1,2-a]pyridine-2-carboxamide and its hydrochloride (1:2)
6-(6-Aminopyridin-2-yl)-N-(thiophen-3-yl)imidazo[1,2-a]pyridine-2-carboxamide and its hydrochloride (1:2)
6-(6-Aminopyridin-2-yl)-N-(isoxazol-3-yl)imidazo[1,2-a]pyridine-2-carboxamide and its hydrochloride (1:2)
6-(6-Aminopyridin-2-yl)-N-(1,3,4-thiadiazol-2-yl)imidazo[1,2-a]pyridine-2-carboxamide and its hydrochloride (1:2)
6-(6-Aminopyridin-2-yl)-N-(isoxazol-4-yl)imidazo[1,2-a]pyridine-2-carboxamide and its hydrochloride (1:2)
N-(Pyridin-2-yl)-6-(1H-pyrrol-3-yl)imidazo[1,2-a]pyridine-2-carboxamide and its trifluoroacetate (1:1)
N-(Isoxazol-3-yl)-6-(1H-pyrrol-3-yl)imidazo[1,2-a]pyridine-2-carboxamide
6-(1H-Pyrrol-3-yl)-N-(1,3,4-thiadiazol-2-yl)imidazo[1,2-a]pyridine-2-carboxamide
N-(Oxazol-2-yl)-6-(1H-pyrrol-3-yl)imidazo[1,2-a]pyridine-2-carboxamide
N-(Isoxazol-4-yl)-6-(1H-pyrrol-3-yl)imidazo[1,2-a]pyridine-2-carboxamide
6-(1H-Pyrazol-3-yl)-N-(pyridin-2-yl)imidazo[1,2-a]pyridine-2-carboxamide
6-(1H-Pyrazol-3-yl)-N-(thiophen-3-yl)imidazo[1,2-a]pyridine-2-carboxamide and its trifluoroacetate (1:1)
6-(1H-Pyrazol-3-yl)-N-(thiazol-2-yl)imidazo[1,2-a]pyridine-2-carboxamide and its trifluoroacetate (1:1)
N-(Isoxazol-3-yl)-6-(1H-pyrazol-3-yl)imidazo[1,2-a]pyridine-2-carboxamide
6-(1H-Pyrazol-3-yl)-N-(1,3,4-thiadiazol-2-yl)imidazo[1,2-a]pyridine-2-carboxamide and its trifluoroacetate (1:1)
N,6-Di(1H-pyrazol-3-yl)imidazo[1,2-a]pyridine-2-carboxamide
6-(Furan-2-yl)-N-(1,3-oxazol-2-yl)imidazo[1,2-a]pyridine-2-carboxamide and its trifluoroacetate (1:1)
6-(Oxazol-2-yl)-N-(1,3,4-thiadiazol-2-yl)imidazo[1,2-a]pyridine-2-carboxamide
6-(Oxazol-2-yl)-N-(1H-pyrazol-3-yl)imidazo[1,2-a]pyridine-2-carboxamide
6-(Furan-3-yl)-N-(1,3-oxazol-2-yl)imidazo[1,2-a]pyridine-2-carboxamide and its trifluoroacetate (1:1)
6-[5-(Hydroxymethyl)furan-2-yl]-N-(1H-pyrazol-3-yl)imidazo[1,2-a]pyridine-2-carboxamide
6-(1H-Imidazol-4-yl)-N-(thiophen-3-yl)imidazo[1,2-a]pyridine-2-carboxamide
6-(1H-Imidazol-4-yl)-N-(1,3-thiazol-2-yl)imidazo[1,2-a]pyridine-2-carboxamide
N-(Pyridin-2-yl)-6-(1H-1,2,3-triazol-4-yl)imidazo[1,2-a]pyridine-2-carboxamide
N-(Thiophen-3-yl)-6-(1H-1,2,3-triazol-4-yl)imidazo[1,2-a]pyridine-2-carboxamide and the acid-addition salts thereof.

In accordance with the invention, the compounds of general formula (I) may be prepared according to the process described in Scheme 1.

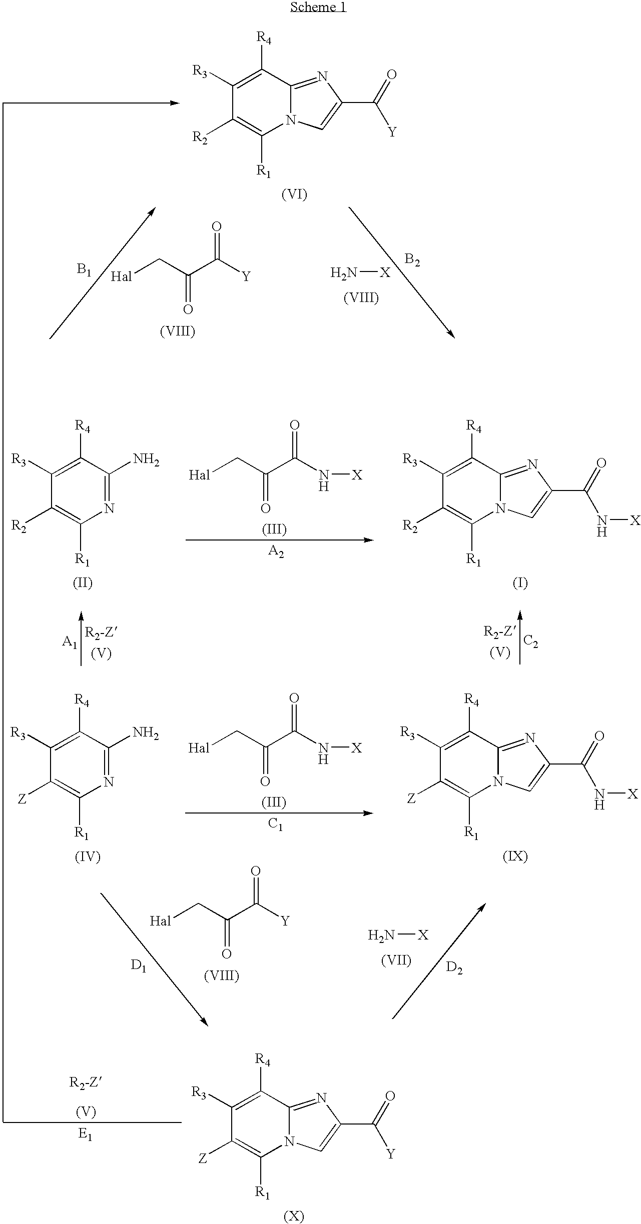

The first synthetic route (transformation A₂) consists in preparing, according to the methods known to those skilled in the art, a 2-aminopyridine of formula (II), in which R₁, R₂, R₃ and R₄ are defined as previously, and then in forming the imidazo[1,2-a]pyridine ring by condensation of a halo derivative of 2-oxopropionamide (III) in which Hal represents a chlorine, bromine or iodine atom and X is defined as previously, by analogy with the methods described by J-J. Bourguignon et al. in Aust. J. Chem., 50, 719 (1997) and by J. G. Lombardino in J. Org. Chem., 30, 2403 (1965), for example.

The halo derivatives of 2-oxo-N-heteroaryl-propionamide (III) may be obtained, for example, according to the method described by R. Kluger et al. in J. Am. Chem. Soc., 106, 4017 (1984).

The 2-aminopyridines of formula (II) in which R₁, R₂, R₃ and R₄ are defined as previously may be prepared, for example, via transformation A₁, i.e.:

via a coupling reaction of a 2-aminopyridine derivative of formula (IV) in which R₁, R₃ and R₄ are defined as previously and Z represents a boryl, stannyl or silyl group, with a derivative R₂—Z′ (V) in which R₂ is defined as previously and Z′ represents a halogen atom such as bromine or iodine or a sulfonyloxy group,

via a coupling reaction of a 2-aminopyridine derivative of formula (IV) in which R₁, R₃ and R₄ are defined as previously and Z represents a halogen atom such as bromine or iodine, with a derivative R₂—Z′ (V) in which R₂ is defined as previously and Z′ represents a reactive group such as a boryl, stannyl or silyl group or a hydrogen atom,

or via any other method known to those skilled in the art.

The second synthetic route (transformation B₂) consists in coupling an imidazopyridine-2-carboxylic acid or a derivative thereof of formula (VI), in which R₁, R₂, R₃ and R₄ are defined as previously, Y represents a hydroxyl, halogen or (C₁-C₆)alkoxy with a heteroarylamine X—NH₂ of formula (VII), in which X is defined as previously, according to methods known to those skilled in the art. Thus, the acid may be converted beforehand into a reactive derivative thereof such as an acid halide, anhydride, mixed anhydride or activated ester, and then reacted with the amine (VII) in the presence of a base such as diisopropylethylamine, triethylamine or pyridine, in an inert solvent such as THF, DMF or dichloromethane. The coupling may also be performed in the presence of a coupling agent such as CDI, EDCI, HATU or HBTU under the same conditions without isolating the reaction intermediate. Alternatively, the amine (VII) may be reacted with an ester of the acid of formula (VI) in the presence of a catalyst such as trimethylaluminium, according to the method of Weinreb, S. et al. (Tet. Lett. (1977), 18, 4171) or zirconium tert-butoxide.

The imidazopyridine-2-carboxylic acid derivatives of formula (VI), in which R₁, R₂, R₃ and R₄ are defined as previously and Y represents a group (C₁-C₆)alkoxy, hydroxyl or a halogen atom are prepared by condensation of a 2-aminopyridine of formula (II), in which R₁, R₂, R₃ and R₄ are defined as previously with a 3-halo-2-oxopropionic acid ester of formula (VIII), in which Hal represents a halogen and Y represents a group (C₁-C₆)alkoxy, under conditions similar to those used for the condensation of a derivative of formula (II) with a derivative of formula (III), followed, where appropriate, by conversion of the ester to the acid and then to the acid chloride or another reactive derivative (transformation B₁).

The third synthetic route (transformation C₂) consists in coupling a derivative of general formula (IX), in which R₁, R₃, R₄ and X are defined as previously and Z represents a halogen atom such as bromine or iodine, a sulfonyloxy group or a reactive group such as boryl, stannyl or silyl, with a derivative of formula R₂—Z′ (V) in which R₂ is defined as previously and

Z′ represents a reactive group such as a boryl, stannyl or silyl group or a hydrogen atom when Z represents a halogen atom or a sulfonyloxy group, or

Z′ represents a halogen atom such as bromine or iodine when Z represents a reactive group such as a boryl, stannyl or silyl group or a hydrogen atom.

The derivatives of general formula (IX), in which R₁, R₃, R₄, X and Z are defined as previously, may be prepared:

by condensation of a 2-aminopyridine of formula (IV), in which R₁, R₃, R₄ and Z are defined as previously, with a 2-oxo-N-heteroaryl-propionamide derivative (III), in which Hal represents a chlorine, bromine or iodine atom and X is defined as previously (transformation C₁), according to methods described for the conversion of a compound of formula (II) into a compound of formula (I) or

by amidation of an imidazopyridine-2-carboxylic acid or a derivative thereof of formula (X) in which R₁, R₂, R₃ and R₄ are defined as previously, Y represents a hydroxyl, a halogen atom or a group (C₁-C₆)alkoxy, with a heteroarylamine X—NH₂ of formula (VII), in which X is defined as previously (transformation D₂), according to methods described for the conversion of a compound of formula (VI) into a compound of formula (I).

The imidazopyridine-2-carboxylic acids or derivatives thereof of formula (X), in which R₁, R₃ and R₄ are defined as previously, Y is (C₁-C₆)alkoxy, OH or halogen and Z represents a boryl, stannyl or silyl group or a halogen atom, may be prepared (transformation D₁) by condensation of a 2-aminopyridine of formula (IV), in which R₁, R₃ and R₄ are defined as previously and Z represents a boryl, stannyl or silyl group or a halogen atom, with a 3-halo-2-oxopropionic acid ester of formula (VIII), in which Hal represents a halogen and Y represents a group (C₁-C₆)alkoxy, under conditions similar to those mentioned previously for the condensation of the 2-aminopyridines of formula (II) with a derivative of formula (VIII), to obtain the imidazopyridine-2-carboxylic acids or derivatives thereof of formula (VI) according to transformation B₁, followed, where appropriate, by conversion of the ester into the acid and then into the acid chloride or another reactive derivative.

The imidazopyridine-2-carboxylic acids or derivatives thereof of formula (VI), in which R₁, R₂, R₃ and R₄ are defined as previously and Y is (C₁-C₆)alkoxy, hydroxyl or halogen, may also be prepared (transformation E₁) by coupling a derivative of general formula (X), in which R₁, R₃, and R₄ are defined as previously, Y represents a group (C₁-C₆)alkoxy and Z represents a halogen atom such as bromine or iodine, a sulfonyloxy group or a reactive group such as boryl, stannyl or silyl, with a derivative of formula R₂—Z′ (V) in which R₂ is defined as previously and

Z′ represents a reactive group such as a boryl, stannyl or silyl group or a hydrogen atom when Z represents a halogen atom or a sulfonyloxy group, or

Z′ represents a halogen atom such as bromine or iodine when Z represents a reactive group such as a boryl, stannyl or silyl group or a hydrogen atom,

followed, where appropriate, by conversion of the ester into the acid and then into the acid chloride or another reactive derivative (transformation E₁).

The coupling of the derivatives of formula (IV), (IX) or (X) with the products of formula (V) may be performed via any method known to those skilled in the art, in particular by working in the presence of copper-based or palladium-based catalysts, or ligands such as phosphines, according to or by analogy with the methods described, for example, in the following references and cited references:

for the reactions of Suzuki type: N. Miyaura, A. Suzuki, Chem. Rev., 95, 2457, (1995),

for the reactions of Stille type: V. Farina et al., Org. React., 50, 1 (1997),

for the reactions of Hiyama type: T. Hiyama et al., Top. Curr. Chem., 2002, 219, 61 (2002),

for the reactions of Negishi type: E. Negishi et al., Chem. Rev., 103, 1979 (2003),

for the reactions of Bellina type: M. Miura et al., Chem. Lett., 200 (2007).

It is also possible to perform the coupling in order to form as intermediates, but without isolating them, organometallic derivatives such as zinc derivatives.

In accordance with the invention, the compounds of general formulae (I), (VI) and (II) may also be prepared according to the processes described in Scheme 2.

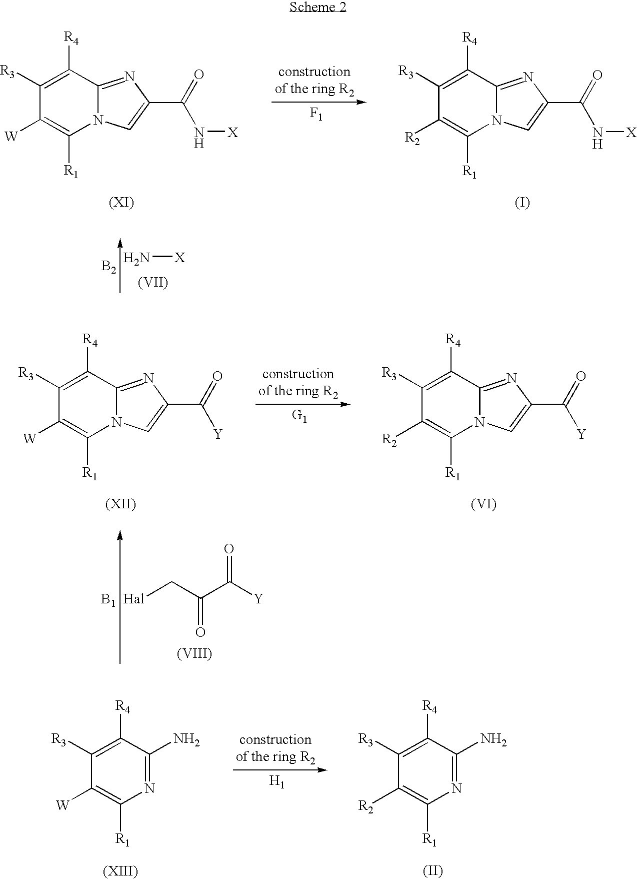

This synthetic route consists in converting a compound of general formula (XI), (XII) or (XIII), in which R₁, R₃, R₄, X and Y are defined as previously and W represents a precursor group for constructing the heterocycle of formula R_{2,} according to the methods known to those skilled in the art.

By way of example, W may represent:

a 2-haloacyl group such as bromoacetyl, or a 1-halo-2-oxoalkyl group such as 1-bromo-2-oxoethyl, which may be converted, for example, into a thiazolyl, imidazolyl or oxazolyl group by treatment with thiourea, thioamide, guanidine, urea or amide derivatives,

an alkynyl group, such as ethynyl, which may be converted into a 1,2,3-triazol-4-yl group,

an acyl group such as formyl, which may be converted, for example, into a 1,3-dioxolanyl-2 or oxazolyl group,

a cyano group, which may be converted, for example, into a dihydroimidazolyl (2) or 1,3,4-triazol-2-yl group.

The compounds of general formula (XI) may be obtained from the compounds of formula (XII) under the conditions described for the preparation of the compounds (I) starting with imidazopyridine-2-carboxylic acid derivatives of formula (VI) via the transformations B₂.

The imidazopyridine-2-carboxylic acid derivatives of general formula (XII) may be obtained from the aminopyridines of formula (XIII), under the conditions described for the conversion of the aminopyridines of formula (II) into compounds of general formula (I), via transformation A₂.

The products of formula (I) and the precursors thereof of formula (II), (IV), (VI), (IX) or (X) may be subjected, if desired and if necessary, in order to obtain products of formula (I) or to be converted into other products of formula (I), to one or more of the following transformation reactions, in any order:
a) an reaction for the esterification or amidation of an acid function,
b) a reaction for the amidation of an amine function,
c) a reaction for the hydrolysis of an ester function to an acid function,
d) a reaction for the transformation of a hydroxyl function into an alkoxy function,
e) a reaction for the oxidation of an alcohol function to an aldehyde or ketone function,
f) a reaction for the reduction of aldehyde or ketone functions to an alcohol function, via reduction or via reaction of an organometallic agent such as an organomagnesium reagent,
g) a reaction for the transformation of a nitrile radical into an aldehyde function,
h) a reaction for the transformation of a nitrile radical into a ketone function,
i) a reaction for the oxidation of an alkenyl group into an aldehyde or ketone function,
j) a reaction for the catalytic coupling of an organometallic derivative such as a boron, tin or silicon derivative with a halo derivative to introduce an alkyl, alkenyl, alkynyl or aryl substituent,
k) a reaction for the conversion of a primary or secondary amino group into a secondary or tertiary amino group via reductive amination or alkylation,
l) a reaction for the protection of reactive functions,
m) a reaction for the removal of the protecting groups that may be borne by the protected reactive functions,
n) a salification reaction with a mineral or organic acid or with a base, to obtain the corresponding salt,
o) a reaction for the resolution of racemic forms into enantiomers,
   the said products of formula (I) thus obtained being, where appropriate, in any possible isomeric form: racemic mixtures, enantiomers and diastereoisomers.

In Scheme 1, the starting compounds and the reagents, when their mode of preparation is not described, are commercially available or are described in the literature, or else may be prepared according to methods that are described therein or that are known to those skilled in the art.

The examples that follow describe the preparation of certain compounds in accordance with the invention. These examples are not limiting, but serve merely to illustrate the present invention. The numbers of the illustrated compounds refer to those given in the table hereinbelow, which illustrates the chemical structures and physical properties of a number of compounds according to the invention.

### EXAMPLE 1

### 6-(6-Aminopyridin-2-yl)-N-(pyridin-2-yl)imidazo[1,2-a]pyridine-2-carboxamide and its hydrochloride (1:2)

276 mg of 6-trimethylstannyl-N-(pyridin-2-yl)imidazo[1,2-a]pyridine-2-carboxamide (intermediate 18), 476 mg of 6-bromo-2-aminopyridine, 111 mg of tetrakis(triphenylphosphine)palladium and 4 mL of N,N-dimethylformamide are placed in a microwave tube. The mixture is heated for 45 minutes in a microwave machine set at 150° C., and then cooled and poured into 100 mL of dichloromethane. The insoluble matter is filtered off and then taken up in a large volume of a methanol, dichloromethane and ethyl acetate mixture until dissolved. The solution is evaporated in the presence of silica and the crude product thus deposited on silica is chromatographed on a silica cartridge, eluting with a 90/10 mixture of dichloromethane and ammoniacal methanol. The fractions containing the expected product are combined and concentrated to dryness under reduced pressure. The residue is triturated with pentane and then filtered off to give 108 mg of 6-(6-aminopyridin-2-yl)-N-(pyridin-2-yl)imidazo[1,2-a]pyridine-2-carboxamide in the form of an off-white solid.

This product is dissolved in boiling dioxane and 150 μL of a 4N solution of hydrogen chloride in dioxane are added to the hot solution. The precipitate is filtered off by suction, rinsed with pentane and then dried to give 130 mg of 6-(6-aminopyridin-2-yl)-N-(pyridin-2-yl)imidazo[1,2-a]pyridine-2-carboxamide hydrochloride (1:2) in the form of an off-white solid.

### EXAMPLE 2

### 6-(1H-Imidazol-4-yl)-N-(pyridin-2-yl)imidazo[1,2-a]pyridine-2-carboxamide

### 2.1: 6-(1-Benzylimidazol-4-yl)-N-(pyridin-2-yl)imidazo[1,2-a]pyridine-2-carboxamide

To a solution of 405 mg of (1-benzylimidazol-4-yl)tributylstannane in 25 mL of toluene are added 300 mg of 6-iodo-N-(pyridin-2-yl)imidazo[1,2-a]pyridine-2-carboxamide (intermediate 17) and 48 mg of tetrakis(triphenylphosphine)palladium(0). The reaction mixture is heated for 3.5 hours at reflux and then concentrated under reduced pressure. The residue is taken up in dichloromethane and washed twice with saturated aqueous potassium fluoride solution. The organic phase is dried and evaporated to dryness under reduced pressure, and the residue is concreted with dichloromethane to give 220 mg of 6-(1-benzylimidazol-4-yl)-N-(pyridin-2-yl)imidazo[1,2-a]pyridine-2-carboxamide in the form of a white solid.

¹H NMR spectrum (DMSO-d6, δ in ppm): 5.26 (s, 2H), 7.18 (m, 1H), 7.30-7.43 (m, 5H), 7.68 (d, J=9.3 Hz, 1H), 7.74-7.80 (m, 2H), 7.88 (m, 1H), 7.91 (d, J=1.0 Hz, 1H), 8.24 (d, J=8.1 Hz, 1H), 8.38 (ddd, J=4.9, 1.9, 1.0 Hz, 1H), 8.63 (s, 1H), 8.99 (broad s, 1H), 9.78 (s, 1H).

Mass spectrum (EI): m/z=395 [M]⁺.

### 2.2: 6-(1H-imidazol-4-yl)-N-(pyridin-2-yl)imidazo[1,2-a]pyridine-2-carboxamide

A mixture of 10 mL of ethanol, 5 mL of dichloromethane, 220 mg of 6-(1-benzylimidazol-4-yl)-N-(pyridin-2-yl)imidazo[1,2-a]pyridine-2-carboxamide, 50 mg of 10% palladium-on-charcoal and 6.8 mL of cyclohexene is heated for 10 minutes in a microwave machine set at 150° C. and again for twice 10 minutes at 150° C. after addition of 20 mg of palladium-on-charcoal and 2 mL of cyclohexene. The reaction mixture is filtered, the insoluble matter is washed with ethanol and the combined filtrates are concentrated to dryness in the presence of silica. The crude product thus deposited on silica is chromatographed on a silica cartridge, eluting with mixtures of dichloromethane and methanol (95/5 and then 90/10). The fractions containing the expected product are combined and concentrated to dryness under reduced pressure to give 13 mg of 6-(1H-imidazol-4-yl)-N-(pyridin-2-yl)imidazo[1,2-a]pyridine-2-carboxamide in the form of an off-white solid.

### EXAMPLE 3

### N,6-Di(pyridin-2-yl)imidazo[1,2-a]pyridine-2-carboxamide

To a mixture of 280 mg of 6-(pyridin-2-yl)imidazo[1,2-a]pyridine-2-carboxylic acid (intermediate 2), 239 mg of 1-hydroxy-7-azabenzotriazole (HOAt), 667 mg of 1-[bis(dimethylamino)methylene]-1H-1,2,3-triazolo[4,5-b]pyridinium 1-oxide hexafluorophosphate (HATU) and 600 μl of diisopropylethylamine in 2 mL of N,N dimethylformamide are added 165 mg of 2-pyridylamine. The reaction mixture is stirred at 20° C. for 16 hours and then filtered. The insoluble matter is washed with water and then taken up in dichloromethane. The organic phase is washed with saturated sodium hydrogen carbonate solution, dried over sodium sulfate and concentrated under reduced pressure. The residue is triturated with methanol to give 70 mg of N,6-di(pyridin-2-yl)imidazo[1,2-a]pyridine-2-carboxamide in the form of a white solid.

### EXAMPLE 4

### N-(Pyridin-2-yl)-6-(thiophen-3-yl)imidazo[1,2-a]pyridine-2-carboxamide

0.55 mL of aqueous 2M potassium carbonate solution and 3 mL of 1,2-dimethoxyethane are placed in a microwave tube and, after degassing with argon, 200 mg of 6-iodo-N-(pyridin-2-yl)imidazo[1,2-a]pyridine-2-carboxamide (intermediate 17), 84 mg of 3-thiophene-boronic acid and 39 mg of dichlorobis(triphenylphosphine)palladium(II) are added. The reaction mixture is heated for 20 minutes in a microwave machine set at 120° C., and then diluted in a mixture of ethyl acetate and water and concentrated. The residue is taken up in a mixture of ethyl ether and water. The solid is washed successively with water (twice), with ether (twice), with a mixture of 2 mL of methanol and 5 mL of ether, then with isopropanol (twice) and finally with methanol. Since the crude product thus obtained is contaminated with about 7% of unreacted 6-iodo-N-(pyridin-2-yl)imidazo[1,2-a]pyridine-2-carboxamide, it is recycled under similar conditions to complete the conversion (mixture of 0.22 mL of 2M potassium carbonate, 3 mL of 1,2-dimethoxyethane, 28 mg of 3-thiopheneboronic acid and 30 mg of dichlorobis(triphenylphosphine)palladium(II) heated for 10 minutes by microwave at 100° C.). The solid obtained after evaporating the reaction mixture is washed successively with water (twice), with methanol (twice), with ether and finally with pentane, and dried to give 103 mg of N-(pyridin-2-yl)-6-(thiophen-3-yl)imidazo[1,2-a]pyridine-2-carboxamide in the form of a light-grey solid.

### EXAMPLE 5

### N-(Isoxazol-4-yl)-6-(pyridin-2-yl)imidazo[1,2-a]pyridine-2-carboxamide

To a suspension of 65 mg of 6-(pyridin-2-yl)imidazo[1,2-a]pyridine-2-carboxylic acid (intermediate 2) and 104 mg of 1-(3-dimethylaminopropyl)-3-ethylcarbodiimide hydrochloride in 2 mL of anhydrous pyridine, placed under argon, are added 68 mg of isoxazol-4-ylamine. The reaction mixture is stirred for 16 hours at 50° C. and then concentrated under reduced pressure. The residue is taken up in dichloromethane and washed with water. The organic phase is dried over magnesium sulfate and concentrated to dryness under reduced pressure to give 49 mg of N-(isoxazol-4-yl)-6-(pyridin-2-yl)imidazo[1,2-a]pyridine-2-carboxamide in the form of a beige-coloured solid.

### EXAMPLES 6 TO 23

### Coupling of the 6-(heterocyclyl)imidazo[1,2-a]pyridine-2-carboxylic acids with the heteroaromatic amines

The compounds of Examples 6 to 23 are obtained by coupling the 6-(heterocyclyl)imidazo[1,2-a]pyridine-2-carboxylic acids (intermediates 1 to 14) with the appropriate heteroaromatic amines according to the procedure of Example 5. If necessary, the product obtained may be repurified by column chromatography on silica.

### EXAMPLE 24

### 6-(1H-Imidazol-4-yl)-N-(isoxazol-3-yl)imidazo[1,2-a]pyridine-2-carboxamide

### 24.1: 6-(1-Triphenylmethyl-1H-imidazol-4-yl)-N-(isoxazol-3-yl)imidazo[1,2-a]pyridine-2-carboxamide

This compound is obtained by coupling 6-(1-triphenylmethyl-1H-imidazol-4-yl)imidazo[1,2-a]pyridine-2-carboxylic acid with isoxazol-3-ylamine according to the procedure of Example 5.

### 24.2: 6-(1H-imidazol-4-yl)-N-(isoxazol-3-yl)imidazo[1,2-a]pyridine-2-carboxamide

To a solution of 137 mg of 6-(1-triphenylmethyl-1H-imidazol-4-yl)-N-(isoxazol-3-yl)imidazo[1,2-a]pyridine-2-carboxamide in 3 mL of ethanol are added 3 mL of aqueous 4N hydrochloric acid solution. The reaction mixture is stirred at 25° C. for 16 hours and then treated with aqueous 2N sodium carbonate solution until a pH of 8-9 is obtained. The residue obtained after evaporation under reduced pressure is chromatographed on silica, eluting with mixtures of dichloromethane and methanol (from 96/4 to 90/10). The fractions containing the expected product are combined and concentrated to dryness under reduced pressure to give 13 mg of 6-(1H-imidazol-4-yl)-N-(isoxazol-3-yl)imidazo[1,2-a]pyridine-2-carboxamide in the form of a beige-coloured solid.

### EXAMPLE 25

### 6-(6-Aminopyridin-2-yl)-N-(thiazol-2-yl)imidazo[1,2-a]pyridine-2-carboxamide

### 25.1: 6-(6-{[(1,1-Dimethylethoxy)carbonyl]amino}pyridin-2-yl)-N-(thiazol-2-yl)imidazo[1,2-a]pyridine-2-carboxamide

This compound is obtained by coupling 6-(6-{[(1,1-dimethylethoxy)carbonyl]amino}-pyridin-2-yl)imidazo[1,2-a]pyridine-2-carboxylic acid with 2-thiazolylamine according to the procedure of Example 5.

### 25.2: 6-(6-Aminopyridin-2-yl)-N-(thiazol-2-yl)imidazo[1,2-a]pyridine-2-carboxamide

To a solution of 124 mg of 6-(6-{[(1,1-dimethylethoxy)carbonyl]amino}pyridin-2-yl)-N-(thiazol-2-yl)imidazo[1,2-a]pyridine-2-carboxamide in 0.5 mL of dioxane cooled to 0° C. are added 1.5 mL of aqueous 4N hydrochloric acid solution. The reaction mixture is stirred at 25° C. for 3 hours and then concentrated to dryness. The solid is washed with 5 mL of ethyl ether, and then taken up in 20 mL of dichloromethane, which is evaporated off under reduced pressure (3 times), and then washed again with 5 mL of ethyl ether and dried to give 114 mg of 6-(6-aminopyridin-2-yl)-N-(thiazol-2-yl)imidazo[1,2-a]pyridine-2-carboxamide in the form of a white solid.

The intermediates described below are useful for preparing the compounds of the present invention.

### Intermediate 1: 6-(6-{[(1,1-Dimethylethoxy)carbonyl]amino}pyridin-2-yl)imidazo[1,2-a]pyridine-2-carboxylic acid

### 1.1: Ethyl 6-(6-aminopyridin-2-yl)imidazo[1,2-a]pyridine-2-carboxylate

350 mg of 2-amino-6-bromopyridine, 750 mg of 2-ethoxycarbonylimidazo[1,2-a]pyridine-6-boronic acid and 57 mg of [1,1′-bis(diphenylphosphino)ferrocene]dichloropalladium are degassed under vacuum and then suspended, under argon, in 20 mL of degassed dioxane. After addition of 2 mL of aqueous 2N sodium carbonate solution, the mixture is degassed under vacuum and then placed under argon and heated for 5 hours at 90° C., then cooled, diluted and stirred in a mixture of 50 mL of saturated sodium bicarbonate solution and 50 mL of dichloromethane. The organic phase is dried over sodium sulfate, filtered and concentrated to dryness under reduced pressure. The residue is chromatographed on silica, eluting with a mixture of ethyl acetate and hexane. The fractions containing the expected product are combined and concentrated to dryness under reduced pressure to give 446 mg of ethyl 6-(6-aminopyridin-2-yl)imidazo[1,2-a]pyridine-2-carboxylate.

¹H NMR spectrum (DMSO-d6, δ in ppm): 9.13 (dd, J=1.0, 1.6 1H), 8.61 (d, J=0.7, 1H), 7.94 (dd, J=1.8, 9.6, 1H), 7.65 (d, J=9.6, 1H), 7.50 (t, J=8.1, 1H), 7.07 (d, J=7.0, 1H), 6.48 (dd, J=0.3, 8.1, 1H), 6.08 (broad s, 2H), 4.33 (q, J=7.1, 2H), 1.33 (t, J=7.1, 3H).

Mass spectrum (APCI): m/z=283 [M+H]⁺.

### 1.2: Ethyl 6-(6-{[(1,1-dimethylethoxy)carbonyl]amino}pyridin-2-yl)imidazo[1,2-a]pyridine-2-carboxylate and ethyl 6-(6-{bis[(1,1-dimethylethoxy)carbonyl]amino}pyridin-2-yl)-imidazo[1,2-a]pyridine-2-carboxylate

To a suspension of 700 mg of ethyl 6-(6-aminopyridin-2-yl)imidazo[1,2-a]pyridine-2-carboxylate and 25 mg of 4-dimethylaminopyridine in 5 mL of acetonitrile are added 1.14 mL of di-tert-butyl dicarbonate. The mixture is stirred for 16 hours at 25° C. and then concentrated. The residue is chromatographed on silica, eluting with a gradient of ethyl acetate and hexane (from 50/50 to 100/0) to give 370 mg of ethyl 6-(6-{bis[(1,1-dimethylethoxy)carbonyl]amino}pyridin-2-yl)imidazo[1,2-a]pyridine-2-carboxylate

¹H NMR spectrum (DMSO-d6, δ in ppm): 9.23 (s, 1H), 8.65 (s, 1H), 8.06-7.98 (m, 2H), 7.95 (d, J=7.7, 1H), 7.76 (d, J=9.6, 1H), 7.43 (d, J=7.8, 1H), 4.33 (q, J=7.0, 2H), 1.43 (s, 18H), 1.34 (t, J=7.1, 3H).

Mass spectrum (APCI): m/z=483 [M+H]⁺.

and 163 mg of ethyl 6-(6-{[(1,1-dimethylethoxy)carbonyl]amino}pyridin-2-yl)imidazo[1,2-a]pyridine-2-carboxylate

¹H NMR spectrum (DMSO-d6, δ in ppm): 9.28 (s, 1H), 8.50 (s, 1H), 8.04-8.00 (m, 2H), 7.95 (d, J=7.8, 1H), 7.70 (d, J=9.6, 1H), 7.38 (d, J=7.9, 1H), 4.31 (q, J=7.0, 2H), 1.39 (s, 9H), 1.33 (t, J=7.1, 3H).

Mass spectrum (APCI): m/z=383 [M+H]⁺.

### 1.3: 6-(6-{[(1,1-Dimethylethoxy)carbonyl]amino}pyridin-2-yl)imidazo[1,2-a]pyridine-2-carboxylic acid

0.9 mL of aqueous 2 M lithium hydroxide solution is added to a solution of 292 mg of ethyl 6-(6-{bis[(1,1-dimethylethoxy)carbonyl]amino}pyridin-2-yl)imidazo[1,2-a]pyridine-2-carboxylate in 4.73 mL of a 50:1 mixture of tetrahydrofuran and methanol. The reaction mixture is stirred for 7 hours at 25° C. and then treated dropwise at 0° C. with 2 N hydrochloric acid until a pH of 3 is obtained. The precipitate formed after 20 minutes is filtered off by suction and washed with water (20 mL) and diethyl ether (20 ml) and then dried under reduced pressure to give 195 mg of 6-(6-{[(1,1-dimethylethoxy)carbonyl]amino}pyridin-2-yl)imidazo[1,2-a]pyridine-2-carboxylic acid in the form of a beige-coloured solid.

¹H NMR spectrum (DMSO-d6, δ in ppm): 13.5-12.0 (br, 1H), 9.80 (s, 1H), 9.24 (s, 1H), 8.51 (s, 1H), 8.03 (dd, J=1.5, 9.6 1H), 7.88 (app, t, J=8.0, 7.8, 1H), 7.77 (d, J=8.2, 1H), 7.73 (d, J=9.6, 1H), 7.62 (d, J=7.5, 1H), 1.50 (s, 9H)

### Intermediate 2: 6-(Pyridin-2-yl)imidazo[1,2-a]pyridine-2-carboxylic acid

### 2.1: Ethyl 6-(pyridin-2-yl)imidazo[1,2-a]pyridine-2-carboxylate

A mixture of 3.18 g of caesium carbonate, 25 mL of dioxane, 9.3 mL of water, 500 mg of 2-iodopyridine, 89 mg of [1,1′-bis(diphenylphosphino)ferrocene]dichloropalladium and 848 mg of ethyl 6-(4,4,5,5-tetramethyl-1,3,2-dioxaborolan-2-yl)imidazo[1,2-a]pyridine-2-carboxylate hydrobromide (1:1) is heated for 2 hours at 110° C., and then partially concentrated, diluted with dichloromethane and filtered. The organic phase is washed with water and dried over magnesium sulfate, filtered and concentrated to dryness under reduced pressure. The residue is chromatographed on a silica cartridge, eluting with a mixture of dichloromethane and cyclohexane (80/20). The fractions containing the expected product are combined and concentrated to dryness under reduced pressure to give 317 mg of ethyl 6-(pyridin-2-yl)imidazo[1,2-a]pyridine-2-carboxylate in the form of a brown oil.

¹H NMR spectrum (DMSO-d6, δ in ppm): 1.34 (t, J=7.0 Hz, 3H), 4.33 (q, J=7.0 Hz, 2H), 7.42 (ddd, J=7.5, 5.5, 2.0 Hz, 1H), 7.73 (d, J=9.3 Hz, 1H), 7.85-8.02 (m, 2H), 8.07 (dd, J=9.3, 2.0 Hz, 1H), 8.64 (s, 1H), 8.70 (broad d, J=5.5 Hz, 1H), 9.36 (broad s, 1H).

Mass spectrum (LC-MS-DAD-ELSD): m/z 268 [M+H]⁺.

### 2.2: 6-(Pyridin-2-yl)imidazo[1,2-a]pyridine-2-carboxylic acid

317 mg of ethyl 6-(pyridin-2-yl)imidazo[1,2-a]pyridine-2-carboxylate are saponified under conditions similar to those described for the preparation of intermediate 1 (step 1.3) to give 280 mg of 6-(pyridin-2-yl)imidazo[1,2-a]pyridine-2-carboxylic acid in the form of a pasty pink solid.

¹H NMR spectrum (DMSO-d6, δ in ppm): 7.47 (m, 1H), 7.83 (d, J=9.8 Hz, 1H), 7.99 (dt, J=8.5, 2.0 Hz, 1H), 8.06 (d, J=8.5 Hz, 1H), 8.31 (broad d, J=9.8 Hz, 1H), 8.73 (m, 2H), 9.52 (broad s, 1H).

Mass spectrum (LC-MS-DAD-ELSD): m/z 240 [M+H]⁺.

### Intermediate 3: 6-(1-Triphenylmethyl-1H-imidazol-4-yl)imidazo[1,2-a]pyridine-2-carboxylic acid

### 3.1: Ethyl 6-(1-triphenylmethyl-1H-imidazol-4-yl)imidazo[1,2-a]pyridine-2-carboxylate

873 mg of 4-iodo-1-triphenylmethylimidazole, 750 mg of 2-ethoxycarbonylimidazo[1,2-a]pyridine-6-boronic acid, 23 mg of palladium acetate and 70 mg of (2-biphenyl)dicyclohexyl-phosphine are degassed under vacuum and then suspended, under argon, in a degassed mixture of 15 mL of toluene, 5 mL of water and 5 mL of N-methylpyrrolidone. After addition of 950 mg of potassium phosphate, the mixture is degassed under vacuum and then placed under argon and heated for 15 minutes at 100° C. by microwave, then cooled, diluted and stirred in a mixture of 50 mL of saturated sodium bicarbonate solution and 50 mL of dichloromethane. The organic phase is dried over sodium sulfate, filtered and concentrated to dryness under reduced pressure. The residue is chromatographed on silica, eluting with a mixture of ethyl acetate and hexane. The fractions containing the expected product are combined and concentrated to dryness under reduced pressure to give 508 mg of ethyl 6-(1-triphenylmethyl-1H-imidazol-4-yl)imidazo[1,2-a]pyridine-2-carboxylate.

¹H NMR spectrum (DMSO-d6, δ in ppm): 8.97 (s, 1H), 8.54 (s, 1H), 7.76-7.72 (m, 1H), 7.56-7.52 (m, 3H), 7.47-7.37 (m, 9H), 7.20-7.17 (m, 6H), 4.31-4.27 (m, 2H), 1.34-1.20 (m, 3H).

Mass spectrum (APCI): m/z=499 [M+H]⁺.

### 3.2: 6-(1-Triphenylmethyl-1H-imidazol-4-yl)imidazo[1,2-a]pyridine-2-carboxylic acid

500 mg of ethyl 6-(1-triphenylmethyl-1H-imidazol-4-yl)imidazo[1,2-a]pyridine-2-carboxylate are saponified under conditions similar to those described for the preparation of intermediate 1 (step 1.3) to give 346 mg of 6-(1-triphenylmethyl-1H-imidazol-4-yl)imidazo[1,2-a]pyridine-2-carboxylic acid.

¹H NMR spectrum (DMSO-d6, δ in ppm): 9.01 (s, 1H), 8.51 (s, 1H), 7.83 (d, J=9.5, 1H), 7.59-7.56 (m, 3H), 7.47-7.37 (m, 9H), 7.20-7.17 (m, 6H). No exchangeable proton is observed

Mass spectrum (APCI): m/z=471 [M+H]⁺.

### Intermediate 4: 6-(2-{[(1,1-Dimethylethoxy)carbonyl]amino}thiazol-4-yl)imidazo[1,2-a]pyridine-2-carboxylic acid

### 4.1: Ethyl 6-(2-{[(1,1-dimethylethoxy)carbonyl]amino}thiazol-4-yl)imidazo[1,2-a]pyridine-2-carboxylate

465 mg of tert-butyl 4-iodothiazol-2-ylcarbamate, 434 mg of 2-ethoxycarbonylimidazo[1,2-a]pyridine-6-boronic acid and 104 mg of [1,1′-bis(diphenylphosphino)ferrocene]dichloropalladium are degassed under vacuum. After addition of 10 mL of degassed tetrahydrofuran and 0.66 mL of aqueous 2N sodium carbonate solution, the reaction mixture is heated for 2 hours at 100° C., then cooled, diluted with dichloromethane and washed with aqueous semi-saturated bicarbonate solution. The organic phase is dried over magnesium sulfate, filtered and concentrated to dryness under reduced pressure. The residue is chromatographed on silica, eluting with a mixture of dichloromethane:methanol (99:1 to 99:2). The fractions containing the expected product are combined and concentrated to dryness under reduced pressure. The solid obtained is washed with 5 mL of diethyl ether to give 125 mg of ethyl 6-(2-{[(1,1-dimethylethoxy)carbonyl]amino}thiazol-4-yl)imidazo[1,2-a]pyridine-2-carboxylate in the form of an off-white solid.

¹H NMR spectrum (DMSO-d6, δ in ppm): 11.65 (s, 1H), 8.84 (s, 1H), 8.47 (s, 1H), 7.84 (s, 1H), 7.68-7.71 (m, 2H), 4.32 (q, J=7.1, 2H), 1.51 (s, 9H), 1.33 (t, J=7.1, 3H).

Mass spectrum (APCI): m/z=389 [M+H]⁺.

### 4.2: 6-(2-{[(1,1-Dimethylethoxy)carbonyl]amino}thiazol-4-yl)imidazo[1,2-a]pyridine-2-carboxylic acid

125 mg of ethyl 6-(2-{[(1,1-dimethylethoxy)carbonyl]amino}thiazol-4-yl)imidazo[1,2-a]pyridine-2-carboxylate are saponified under conditions similar to those described for the preparation of intermediate 1 (step 1.3) to give 90 mg of 6-(2-{[(1,1-dimethylethoxy)carbonyl]amino}thiazol-4-yl)imidazo[1,2-a]pyridine-2-carboxylic acid in the form of a brown solid.

¹H NMR spectrum (DMSO-d6, δ in ppm): 11.66 (s, 1H), 8.86 (s, 1H), 8.42 (s, 1H), 7.84 (s, 1H), 7.67-7.69 (m, 2H), 1.51 (s, 9H).

Mass spectrum (APCI): m/z=361 [M+H]⁺.

### Intermediate 5: 6-(1H-Pyrrol-3-yl)imidazo[1,2-a]pyridine-2-carboxylic acid

### 5.1 Ethyl 6-[1-(triisopropylsilyl)-1H-pyrrol-3-yl]imidazo[1,2-a]pyridine-2-carboxylate

100 mg of ethyl 6-iodo-imidazo[1,2-a]pyridine-2-carboxylate, 135 mg of 1-(triisopropylsilyl)pyrrole-3-boronic acid and 18 mg of tetrakis(triphenylphosphine)palladium(0) are degassed under vacuum and then suspended, under argon, in a degassed mixture of 1.5 mL of 1,2-dimethoxyethane, 1.5 mL of ethanol and 316 μl of aqueous 2N sodium carbonate solution. The reaction mixture is heated at reflux for 4 hours, then cooled, diluted and stirred with a mixture of 5 mL of aqueous semi-saturated sodium bicarbonate solution and 5 mL of dichloromethane. The organic phase is dried over sodium sulfate, filtered and concentrated to dryness under reduced pressure. The residue is chromatographed on silica, eluting with a mixture of ethyl acetate and hexane (50/50). The fractions containing the expected product are combined and concentrated to dryness under reduced pressure to give 121 mg of ethyl 6-[1-(triisopropylsilyl)-1H-pyrrol-3-yl]imidazo[1,2-a]pyridine-2-carboxylate.

¹H NMR spectrum (DMSO-d6, δ in ppm): 8.76 (s, 1H), 8.42 (s, 1H), 7.70 (dd, J=1.9, 9.7 1H), 7.59 (d, J=9.7 1H), 7.37 (broad s, 1H), 6.94 (m, 1H), 6.63 (m, 1H), 4.33 (q, J=6.9, 2H), 1.61-1.50 (m, 3H), 1.33 (t, J=6.9, 3H), 1.10-1.03 (m, 18H).

Mass spectrum (APCI): m/z=412 [M+H]⁺.

### 5.2: 6-(1H-Pyrrol-3-yl)imidazo[1,2-a]pyridine-2-carboxylic acid hydrochloride (1:1)

292 mg of ethyl 6-[1-(triisopropylsilyl)-1H-pyrrol-3-yl]imidazo[1,2-a]pyridine-2-carboxylate are saponified under conditions similar to those described for the preparation of intermediate 1 (step 1.3) to give 140 mg of 6-(1H-pyrrol-3-yl)imidazo[1,2-a]pyridine-2-carboxylic acid hydrochloride (1:1) in the form of a white solid.

¹H NMR spectrum (DMSO-d6, δ in ppm): 11.07 (broad s, 1H), 8.73 (s, 1H), 8.39 (s, 1H), 7.69 (dd, J=1.3, 9.5, 1H), 7.59 (d, J=9.5, 1H), 7.31 (s, 1H), 6.86 (s, 1H), 6.46 (s, 1H).

Mass spectrum (APCI): m/z=228 [M+H]⁺.

### Intermediate 6: 6-(1H-Pyrazol-3-yl)imidazo[1,2-a]pyridine-2-carboxylic acid

### 6.1: Ethyl 6-(1H-pyrazol-3-yl)imidazo[1,2-a]pyridine-2-carboxylate

This product is prepared under conditions similar to those described for the preparation of intermediate 5 (step 5.1), replacing the 1-(triisopropylsilyl)pyrrole-3-boronic acid with pyrazole-3-boronic acid.

¹H NMR spectrum (MeOD-d4, δ in ppm): 8.89 (t, J=1.2 and 2.4, 1H), 8.45 (d, J=0.6, 1H), 7.89 (d, J=9.0, 1H), 7.76 (broad s, 1H), 7.67 (d, J=9.5, 1H), 6.77 (d, J=2.4, 1H), 4.42 (q, J=7.1, 2H), 1.43 (t, J=7.1, 3H).

Mass spectrum (APCI): m/z=257 [M+H]⁺.

### 6.2: 6-(1H-Pyrazol-3-yl)imidazo[1,2-a]pyridine-2-carboxylic acid

128 mg of ethyl 6-(1H-pyrazol-3-yl)imidazo[1,2-a]pyridine-2-carboxylate are saponified under conditions similar to those described for the preparation of intermediate 1 (step 1.3) to give 113 mg of 6-(1H-pyrazol-3-yl)imidazo[1,2-a]pyridine-2-carboxylic acid.

¹H NMR spectrum (DMSO-d6, δ in ppm): 13.50-12.50 (broad s, 1H), 9.03 (s, 1H), 8.40 (s, 1H), 7.83-7.80 (m, 2H), 7.63 (d, J=9.4, 1H), 6.74 (s, 1H).

### Intermediate 7: 6-(1H-Pyrazol-4-yl)imidazo[1,2-a]pyridine-2-carboxylic acid

### 7.1: Ethyl 6-(1H-pyrazol-4-yl)imidazo[1,2-a]pyridine-2-carboxylate

This product is prepared under conditions similar to those described for the preparation of intermediate 5 (step 5.1), replacing the 1-(triisopropylsilyl)pyrrole-3-boronic acid with pyrazole-4-boronic acid and heating at 90° C. by microwave for 37 minutes.

¹H NMR spectrum (DMSO-d6, δ in ppm): 13.10 (broad s, 1H), 8.83 (s, 1H), 8.43 (s, 1H), 8.25 (broad s, 1H), 7.94 (broad s, 1H), 7.69-7.61 (m, 2H), 4.31 (q, J=7.1, 2H), 1.32 (t, J=7.1, 3H).

Mass spectrum (APCI): m/z=257 [M+H]⁺.

### 7.2: 6-(1H-Pyrazol-4-yl)imidazo[1,2-a]pyridine-2-carboxylic acid

128 mg of ethyl 6-(1H-pyrazol-4-yl)imidazo[1,2-a]pyridine-2-carboxylate are saponified under conditions similar to those described for the preparation of intermediate 1 (step 1.3) to give 60 mg of 6-(1H-pyrazol-4-yl)imidazo[1,2-a]pyridine-2-carboxylic acid.

¹H NMR spectrum (DMSO-d6, δ in ppm): 14.0-12.0 (broad s, 1H), 8.84 (s, 1H), 8.36 (s, 1H), 8.10 (s, 2H), 7.64 (s, 2H).

### Intermediate 8: 6-(Furan-2-yl)imidazo[1,2-a]pyridine-2-carboxylic acid

### 8.1: Ethyl 6-furan-2-yl)imidazo[1,2-a]pyridine-2-carboxylate

This product is prepared under conditions similar to those described for the preparation of intermediate 5 (step 5.1), replacing the 1-(triisopropylsilyl)pyrrole-3-boronic acid with furan-2-boronic acid.

¹H NMR spectrum (DMSO-d6, δ in ppm): 8.78 (s, 1H), 8.44 (s, 1H), 7.72 (dd, J=1.8, 9.6, 1H), 7.63-7.60 (m, 2H), 6.89 (d, J=3.4, 1H), 6.57 (dd, J=1.8, 3.4, 1H), 4.42 (q, J=7.1, 2H), 1.42 (t, J=7.1, 3H).

Mass spectrum (APCI): m/z=257 [M+H]⁺.

### 8.2: 6-Furan-2-yl)imidazo[1,2-a]pyridine-2-carboxylic acid

384 mg of ethyl 6-furan-2-yl)imidazo[1,2-a]pyridine-2-carboxylate are saponified under conditions similar to those described for the preparation of intermediate 1 (step 1.3) to give 256 mg of 6-furan-2-yl)imidazo[1,2-a]pyridine-2-carboxylic acid.

¹H NMR spectrum (DMSO-d6, δ in ppm): 8.86 (s, 1H), 8.38 (s, 1H), 7.80 (dd, J=1.7, 9.5, 1H), 7.67-7.64 (m, 2H), 6.90 (d, J=3.4, 1H), 6.60 (dd, J=1.8, 3.4, 1H).

### Intermediate 9: 6-(Furan-3-yl)imidazo[1,2-a]pyridine-2-carboxylic acid

### 9.1: Ethyl 6-furan-3-yl)imidazo[1,2-a]pyridine-2-carboxylate

This product is prepared under conditions similar to those described for the preparation of intermediate 5 (step 5.1), replacing the 1-(triisopropylsilyl)pyrrole-3-boronic acid with furan-3-boronic acid.

¹H NMR spectrum (DMSO-d6, δ in ppm): 8.86 (s, 1H), 8.45 (s, 1H), 8.28 (s, 1H), 7.82 (s, 1H), 7.66 (s, 2H), 6.95 (s, 1H), 4.31 (q, J=7.1, 2H), 1.33 (t, J=7.1, 3H).

Mass spectrum (APCI): m/z=257 [M+H]⁺.

### 9.2: 6-Furan-3-yl)imidazo[1,2-a]pyridine-2-carboxylic acid

384 mg of ethyl 6-furan-3-yl)imidazo[1,2-a]pyridine-2-carboxylate are saponified under conditions similar to those described for the preparation of intermediate 1 (step 1.3) to give 287 mg of 6-furan-3-yl)imidazo[1,2-a]pyridine-2-carboxylic acid.

¹H NMR spectrum (DMSO-d6, δ in ppm): 8.86 (s, 1H), 8.38 (s, 1H), 8.27 (s, 1H), 7.81 (s, 1H), 7.64 (s, 2H), 6.95 (s, 1H).

Mass spectrum (APCI): m/z=229 [M+H]⁺.

### Intermediate 10: 6-[5-(Hydroxymethyl)furan-2-yl]imidazo[1,2-a]pyridine-2-carboxylic acid

### 10.1: Ethyl 6-(5-formylfuran-2-yl)imidazo[1,2-a]pyridine-2-carboxylate

2 g of ethyl 6-iodoimidazo[1,2-a]pyridine-2-carboxylate, 1.42 g of 5-formylfuran-2-boronic acid and 231 mg of [1,1′-bis(diphenylphosphino)ferrocene]dichloropalladium are degassed under vacuum and then suspended, under argon, in a degassed mixture of 30 mL of dioxane and 9.4 mL of aqueous 2N sodium carbonate solution. The reaction mixture is heated for 5 hours at 90° C., then stirred for 16 hours at 20° C. and concentrated to dryness. The residue is chromatographed on silica, eluting with a mixture of ethyl acetate and hexane (90/10), with ethyl acetate and then with a mixture (99/1) of ethyl acetate and methanol. The fractions containing the expected product are combined and concentrated to dryness under reduced pressure to give 884 mg of ethyl 6-(5-formylfuran-2-yl)imidazo[1,2-a]pyridine-2-carboxylate.

¹H NMR spectrum (DMSO-d6, δ in ppm): 9.64 (s, 1H), 9.20 (s, 1H), 8.66 (s, 1H), 7.86-7.74 (m, 2H), 7.72 (d, J=3.8, 1H), 7.37 (d, J=3.8, 1H), 4.33 (q, J=7.0, 2H), 1.33 (t, J=7.1, 3H).

Mass spectrum (APCI): m/z=285 [M+H]⁺.

### 10.2: Ethyl 6-[5-(hydroxymethyl)furan-2-yl]imidazo[1,2-a]pyridine-2-carboxylate

To a suspension of 770 mg of ethyl 6-(5-formylfuran-2-yl)imidazo[1,2-a]pyridine-2-carboxylate in 15 mL of ethanol are added 123 mg of sodium borohydride. The reaction mixture is stirred at 25° C. for 90 minutes and then diluted and stirred with 10 mL of dichloromethane and 3 mL of aqueous semi-saturated sodium carbonate solution. The organic phase is separated out, dried over magnesium sulfate, filtered and concentrated under reduced pressure. The residue is chromatographed on silica, eluting with a mixture of dichloromethane and methanol (98/2). The fractions containing the expected product are combined and concentrated to dryness under reduced pressure. The solid obtained is triturated in 5 mL of dichloromethane, filtered off and dried to give 403 mg of ethyl 6-(5-formylfuran-2-yl)imidazo[1,2-a]pyridine-2-carboxylate in the form of a white solid.

¹H NMR spectrum (DMSO-d6, δ in ppm): 8.89 (s, 1H), 8.60 (s, 1H), 7.70 (m, 2H), 6.98 (d, J=3.3, 1H), 6.45 (d, J=3.3, 1H), 5.30 (t, J=5.3, 1H), 4.47 (d, J=5.6, 2H), 4.32 (q; J=7.1, 2H), 1.32 (t, J=7.1, 3H).

Mass spectrum (APCI): m/z=287 [M+H]⁺.

### 10.3: 6-[5-(Hydroxymethyl)furan-2-yl]imidazo[1,2-a]pyridine-2-carboxylic acid

400 mg of ethyl 6-[5-(hydroxymethyl)furan-2-yl]imidazo[1,2-a]pyridine-2-carboxylate are saponified under conditions similar to those described for the preparation of intermediate 1 (step 1.3) to give 346 mg of 6-[5-(hydroxymethyl)furan-2-yl]imidazo[1,2-a]pyridine-2-carboxylic acid in the form of a white solid.

¹H NMR spectrum (DMSO-d6, δ in ppm): 9.06 (s, 1H), 8.73 (s, 1H), 8.03 (d, J=9.5, 1H), 7.82 (d, J=9.5, 1H), 7.09 (d, J=3.3, 1H), 6.49 (d, J=3.2, 1H), 4.49 (s, 2H).

Mass spectrum (APCI): m/z=259 [M+H]⁺.

### Intermediate 11: 6-(Thiophen-3-yl)imidazo[1,2-a]pyridine-2-carboxylic acid

### 11.1: Ethyl 6-(thiophen-3-yl)imidazo[1,2-a]pyridine-2-carboxylate

This product is prepared under conditions similar to those described for the preparation of intermediate 5 (step 5.1), replacing the 1-(triisopropylsilyl)pyrrole-3-boronic acid with thiophene-3-boronic acid (catalyst: dichlorobis(triphenylphosphine)palladium.

¹H NMR spectrum (DMSO-d6, δ in ppm): 1.34 (d, J=7.1 Hz, 3H), 4.32 (q, J=7.1 Hz, 2H), 7.56 (dd, J=5.0, 1.4 Hz, 1H), 7.68 (d, J=9.8 Hz, 1H), 7.73 (dd, J=5.0, 3.0 Hz, 1H), 7.78 (dd, J=9.8, 1.8 Hz, 1H), 7.97 (dd, J=3.0, 1.4 Hz, 1H), 8.48 (s, 1H), 8.98 (broad s, 1H).

Mass spectrum (LC-MS-DAD-ELSD): m/z 273 [M+H]⁺.

### 11.2: 6-(Thiophen-3-yl)imidazo[1,2-a]pyridine-2-carboxylic acid

310 mg of ethyl 6-(thiophen-3-yl)imidazo[1,2-a]pyridine-2-carboxylate are saponified under conditions similar to those described for the preparation of intermediate 1 (step 1.3) to give 250 mg of 6-(thiophen-3-yl)imidazo[1,2-a]pyridine-2-carboxylic acid.

¹H NMR spectrum (DMSO-d6, δ in ppm): 7.57 (d, J=5.4 Hz, 1H), 7.66 (d, J=9.8 Hz, 1H), 7.73 (dd, J=5.4, 2.8 Hz, 1H), 7.76 (dd, J=9.8, 2.0 Hz, 1H), 7.97 (broad d, J=2.0 Hz, 1H), 8.41 (s, 1H), 8.99 (broad s, 1H).

Mass spectrum (LC-MS-DAD-ELSD): m/z 245 [M+H]⁺.

### Intermediate 12: 6-(Oxazol-2-yl)imidazo[1,2-a]pyridine-2-carboxylic acid

### 12.1: Ethyl 6-(oxazol-2-yl)imidazo[1,2-a]pyridine-2-carboxylate

1 g of ethyl 6-iodoimidazo[1,2-a]pyridine-2-carboxylate, 350 mg of tetrakis(triphenylphosphine)palladium(0) and 360 mg of lithium chloride are degassed under vacuum and then suspended, under argon, in 15 mL of degassed dioxane. After addition of 5 g of 2-(tri-n-butylstannyl)oxazole, the reaction mixture is heated at 90° C. for 3.5 hours, then cooled, diluted and stirred with a mixture of 100 mL of aqueous 1M potassium fluoride solution and 200 mL of ethyl acetate. The aqueous phase is extracted with 200 mL of ethyl acetate and the combined organic phases are washed with brine and dried over sodium sulfate, filtered and concentrated to dryness under reduced pressure. The residue is chromatographed on silica, eluting with a gradient of ethyl acetate and hexane (from 80/20 to 100/0). The fractions containing the expected product are combined and concentrated to dryness under reduced pressure to give 530 mg of ethyl 6-(oxazol-2-yl)imidazo[1,2-a]pyridine-2-carboxylate in the form of a yellow powder.

¹H NMR spectrum (DMSO-d6, δ in ppm): 9.30 (d, J=0.8, 1H), 8.68 (s, 1H), 8.30 (s, 1H), 7.85 (dd, J=1.7, 9.5, 1H), 7.79 (d, J=9.5, 1H), 7.44 (d, J=0.6, 1H), 4.33 (q, J=7.0, 2H), 1.33 (t, J=7.1, 3H).

Mass spectrum (APCI): m/z=258 [M+H]⁺.

### 12.2: 6-(Oxazol-2-yl)imidazo[1,2-a]pyridine-2-carboxylic acid

512 mg of ethyl 6-(oxazol-2-yl)imidazo[1,2-a]pyridine-2-carboxylate are saponified under conditions similar to those described for the preparation of intermediate 1 (step 1.3) to give 365 mg of 6-(oxazol-2-yl)imidazo[1,2-a]pyridine-2-carboxylic acid in the form of a white solid.

¹H NMR spectrum (DMSO-d6, δ in ppm): 9.41 (s, 1H), 8.73 (s, 1H), 8.34 (s, 1H), 8.05 (dd, J=1.5, 9.5, 1H), 7.86 (d, J=9.5, 1H), 7.48 (s, 1H).

### Intermediate 13: 6-(1H-1,2,4-Triazol-3-yl)imidazo[1,2-a]pyridine-2-carboxylic acid

### 13.1: Ethyl 6-[ethoxy(imino)methyl]imidazo[1,2-a]pyridine-2-carboxylate

470 mg of sodium ethanethiolate are added to a solution of 1 g of ethyl 6-cyanoimidazo[1,2-a]pyridine-2-carboxylate (J. Med. Chem. (1998), 41(22), 4317) in a mixture of 15 mL of ethanol and 10 mL of dichloromethane cooled to 0° C. The reaction mixture is stirred for 5 hours at 25° C. and filtered, and the filtrate is evaporated to dryness. The residue is chromatographed on silica, eluting with a mixture of dichloromethane and methanol (98/2) to give 625 mg of ethyl 6-[ethoxy(imino)methyl]imidazo[1,2-a]pyridine-2-carboxylate in the form of a pale yellow solid.

¹H NMR spectrum (DMSO-d6, δ in ppm): 9.17 (s, 1H), 9.04 (s, 1H), 8.64 (s, 1H), 7.84 (m, 1H), 7.68 (m, 1H), 4.33 (q, J=7.1, 4H), 1.34 (t=7.2, 6H).

Mass spectrum (APCI): m/z=262 [M+H]⁺.

### 13.2: Ethyl 6-[hydrazino(imino)methyl]imidazo[1,2-a]pyridine-2-carboxylate

To a solution of 625 mg of ethyl 6-[ethoxy(imino)methyl]imidazo[1,2-a]pyridine-2-carboxylate in 12 mL of ethanol is added dropwise at 0-5° C. 0.2 mL of hydrazine hydrate. The reaction mixture is stirred for 2 hours, 73 μL of hydrazine hydrate are then added and the mixture is stirred for a further 2 hours, while allowing the temperature to rise to 25° C. The reaction mixture is concentrated to dryness under reduced pressure and the residue is dried to give 600 mg of ethyl 6-[hydrazino(imino)methyl]imidazo[1,2-a]pyridine-2-carboxylate, which is used in the following synthesis without further purification.

¹H NMR spectrum (DMSO-d6, δ in ppm): 8.77 (broad s, 1H), 8.49 (s, 1H), 7.70 (m, 1H), 7.53 (d, J=9.6, 1H), 5.67 (s, 2H), 5.15 (broad s, 2H), 4.33 (q, J=7.1, 2H), 1.32 (t=7.1, 3H).

Mass spectrum (APCI): m/z=248 [M+H]⁺.

### 13.3: Ethyl 6-(1H-1,2,4-triazol-3-yl)imidazo[1,2-a]pyridine-2-carboxylate

A suspension of 580 mg of ethyl 6-[hydrazino(imino)methyl]imidazo[1,2-a]pyridine-2-carboxylate in 6 mL of formic acid is heated for 20 hours at 85° C. The reaction mixture is concentrated to less than 20% of its initial volume and diluted with 20 mL of water. Solid sodium carbonate is added at 0-5° C. until a pH of 8-9 is obtained. The precipitate is filtered off by suction and then purified by chromatography on silica, eluting with a mixture of dichloromethane and methanol (98/2) to give 320 mg of ethyl 6-(1H-1,2,4-triazol-3-yl)imidazo[1,2-a]pyridine-2-carboxylate.

¹H NMR spectrum (DMSO-d6, δ in ppm): 14.5-14.0 (broad s, 1H), 9.25 (s, 1H), 8.69 (s, 1H), 8.63 (broad s, 1H), 7.94 (dd, J=9.5, 1.5, 1H), 7.73 (d, J=9.5, 1H), 4.33 (q, J=7.0, 2H), 1.33 (t=7.0, 3H)

Mass spectrum (APCI): m/z=258 [M+H]⁺.

### 13.4: 6-(1H-1,2,4-Triazol-3-yl)imidazo[1,2-a]pyridine-2-carboxylic acid

320 mg of ethyl 6-(1H-1,2,4-triazol-3-yl)imidazo[1,2-a]pyridine-2-carboxylate are saponified under conditions similar to those described for the preparation of intermediate 1 (step 1.3) to give 238 mg of 6-(1H-1,2,4-triazol-3-yl)imidazo[1,2-a]pyridine-2-carboxylic acid in the form of an off-white solid.

¹H NMR spectrum (DMSO-d6, δ in ppm): 14.5-14.2 (broad s, 1H), 9.26 (s, 1H), 8.66-8.62 (m, 2H), 7.91 (d, J=9.1, 1H), 7.73 (d, J=9.6, 1H).

### Intermediate 14: 6-(1H-1,2,3-Triazol-4-yl)imidazo[1,2-a]pyridine-2-carboxylic acid

### 14.1: Ethyl 6-[(trimethylsilyl)ethynyl]imidazo[1,2-a]pyridine-2-carboxylate

A mixture of 4 g of ethyl 6-iodoimidazo[1,2-a]pyridine-2-carboxylate, 2.63 mL of ethynyltrimethylsilane and 888 mg of dichlorobis(triphenylphosphine)palladium is degassed under vacuum. 240 mg of degassed N,N-dimethylformamide and 3.52 mL of triethylamine are added. The reaction mixture is degassed under argon and then stirred at 50° C. for 50 hours, cooled and diluted with 20 mL of water. The precipitate is filtered off by suction and washed with 5 mL of water and then chromatographed on silica, eluting with mixtures of ethyl acetate and hexane (from 50/50 to 90/10). The fractions containing the expected product are combined and concentrated to dryness under reduced pressure to give 3.6 g of ethyl 6-[(trimethylsilyl)ethynyl]imidazo[1,2-a]pyridine-2-carboxylate in the form of an off-white solid.

¹H NMR spectrum (DMSO-d6, δ in ppm): 8.61 (s, 1H), 8.22 (s, 1H), 7.36 (d, J=9.5, 1H), 7.07 (dd, J=9.5, 1.7, 1H), 4.07 (q, J=7.1, 2H), 1.08 (t, J=7.1, 3H), 0.01 (s, 9H).

Mass spectrum (APCI): m/z=287 [M+H]⁺.

### 14.2: Ethyl 6-ethynylimidazo[1,2-a]pyridine-2-carboxylate

To a solution of 500 mg of ethyl 6-[(trimethylsilyl)ethynyl]imidazo[1,2-a]pyridine-2-carboxylate in 10 mL of anhydrous tetrahydrofuran, cooled to 0° C., are added dropwise 1.58 mL of a 1M solution of tetrabutylammonium fluoride in tetrahydrofuran. The reaction mixture is stirred for 30 minutes, 5 mL of water are then added and the resulting mixture is extracted 3 times with 20 mL of dichloromethane. The product is purified by chromatography on silica, eluting with mixtures of ethyl acetate and hexane (from 1/3 to 1/1). The fractions containing the expected product are combined and concentrated to dryness under reduced pressure to give 280 mg of ethyl 6-ethynylimidazo[1,2-a]pyridine-2-carboxylate in the form of a yellow solid.

¹H NMR spectrum (DMSO-d6, δ in ppm): 8.86 (d, J=1.0, 1H), 8.50 (d, J=0.6, 1H), 7.63 (d, J=9.4, 1H), 7.37 (d, J=1.7, 9.4, 1H), 4.32 (m, 3H), 1.32 (t, J=7.1 Hz, 3H).

Mass spectrum (APCI): m/z=215 [M+H]⁺.

### 14.3: Ethyl 6-(1H-1,2,3-triazol-4-yl)imidazo[1,2-a]pyridine-2-carboxylate

To a solution of 220 mg of ethyl 6-ethynylimidazo[1,2-a]pyridine-2-carboxylate and 0.21 mL of azidotrimethylsilane in 4 mL of a mixture (9/1) of N,N-dimethylformamide and methanol are added 9.8 mg of cuprous iodide. The reaction mixture is stirred for 2 hours at 100° C. and then cooled, diluted with 4 mL of dichloromethane, filtered through alumina and concentrated to dryness. The residue is chromatographed on silica, eluting with a mixture of dichloromethane and ethanol (97/3). The fractions containing the expected product are combined and concentrated to dryness under reduced pressure to give 125 mg of ethyl 6-(1H-1,2,3-triazol-4-yl)imidazo[1,2-a]pyridine-2-carboxylate in the form of an off-white solid.

¹H NMR spectrum (DMSO-d6, δ in ppm): 15.5-15.0 (broad s, 1H), 9.14 (dd, J=1.1, 1.5, 1H), 8.60 (d, J=0.5, 1H), 8.40 (broad s, 1H), 7.82 (dd, J=1.7, 9.5, 1H), 7.75 (d, J=9.5, 1H), 4.33 (q, J=7.1, 2H), 1.33 (t, J=7.1, 3H).

Mass spectrum (APCI): m/z=258 [M+H]⁺.

### 14.4: 6-(1H-1,2,3-Triazol-4-yl)imidazo[1,2-a]pyridine-2-carboxylic acid

125 mg of ethyl 6-(1H-1,2,3-triazol-3-yl)imidazo[1,2-a]pyridine-2-carboxylate are saponified under conditions similar to those described for the preparation of intermediate 1 (step 1.3) to give 72 mg of 6-(1H-1,2,3-triazol-3-yl)imidazo[1,2-a]pyridine-2-carboxylic acid in the form of a white solid.

¹H NMR spectrum (DMSO-d6, δ in ppm): 16.0-15.0 (broad s, 1H), 9.23 (s, 1H), 8.62 (s, 1H), 8.46 (broad s, 1H), 7.96 (dd, J=1.4, 9.5, 1H), 7.80 (d, J=9.5, 1H).

### Intermediate 15: 6-Iodo-N-(isoxazol-4-yl)imidazo[1,2-a]pyridine-2-carboxamide

To a suspension of 200 mg of 6-iodoimidazo[1,2-a]pyridine-2-carboxylic acid and 266 mg of 1-(3-dimethylaminopropyl)-3-ethylcarbodiimide hydrochloride in 1 mL of anhydrous pyridine are added 175 mg of isoxazol-4-ylamine. The reaction mixture is stirred for 16 hours at 50° C. and then concentrated under reduced pressure. The residue is taken up in 10 mL of a mixture of chloroform and water (1/1). The solid is triturated with 3 mL of water, filtered off by suction and washed with 3 mL of water and then with 3 mL of ethyl ether, and dried to give 280 mg of 6-iodo-N-(isoxazol-4-yl)imidazo[1,2-a]pyridine-2-carboxamide in the form of a beige-coloured solid.

¹H NMR spectrum (DMSO-d6, δ in ppm): 10.93 (broad s, 1H), 9.24 (broad s, 1H), 9.02 (broad s, 1H), 8.81 (broad s, 1H), 8.43 (broad s, 1H), 7.60-7.48 (m, 2H).

Mass spectrum (APCI): m/z=258 [M+H]⁺.

### Intermediate 16: 6-Iodo-N-(thiazol-2-yl)imidazo[1,2-a]pyridine-2-carboxamide

This product is prepared under conditions similar to those described for the preparation of intermediate 15, replacing the isoxazol-4-ylamine with thiazol-2-ylamine.

¹H NMR spectrum (DMSO-d6, δ in ppm): 11.95 (br s, 1H), 9.04 (br s, 1H), 8.59 (br s, 1H), 7.60-7.51 (m, 3H), 7.30 (d, J=3.4, 1H).

Mass spectrum (APCI): m/z=371 [M+H]⁺.

### Intermediate 17: 6-Iodo-N-(pyridin-2-yl)imidazo[1,2-a]pyridine-2-carboxamide

A suspension of 1 g of ethyl 6-iodoimidazo[1,2-a]pyridine-2-carboxylate, 330 mg of 2-pyridylamine, 92 mg of 1-hydroxy-7-azabenzotriazole (HOAt) and 787 mg zirconium tert-butoxide in 12 mL of toluene is stirred for 16 hours at room temperature and then refluxed for 6 hours. After cooling, the medium is diluted with ethyl acetate and filtered. On the one hand, the solid is taken up in dichloromethane and saturated aqueous sodium hydrogen carbonate solution. On the other hand, the filtrate is concentrated to dryness and then taken up in water and dichloromethane, and the organic phase is separated out, dried and concentrated to dryness. The solids obtained from the two sources are combined and triturated with dichloromethane to give 1.42 g of 6-iodo-N-(pyridin-2-yl)imidazo[1,2-a]pyridine-2-carboxamide in the form of a pale yellow solid.

¹H NMR spectrum (DMSO-d6, δ in ppm): 7.19 (dd, J=5.0 and 8.0 Hz, 1H), 7.55 (d, J=9.5 Hz, 1H), 7.60 (dd, J=2.0 and 9.5 Hz, 1H), 7.89 (dt, J=2.0 and 8.0 Hz, 1H), 8.22 (d, J=8.0 Hz, 1H), 8.38 (dd, J=2.0 and 5.0 Hz, 1H), 8.51 (s, 1H), 9.01 (broad s, 1H), 9.79 (s, 1H).

Mass spectrum (IC): m/z 365 [M+H]⁺.

### Intermediate 18: 6-Trimethylstannyl-N-(pyridin-2-yl)imidazo[1,2-a]pyridine-2-carboxamide

To a suspension of 300 mg of 6-iodo-N-(pyridin-2-yl)imidazo[1,2-a]pyridine-2-carboxamide in 16 mL of toluene are added 423 μL of hexamethyldistannane and 50 mg of tetrakis(triphenylphosphine)palladium(0). The reaction mixture is heated for 2 hours at reflux and then stirred for 16 hours at room temperature and filtered off. The filtrate is concentrated under reduced pressure and the residue is chromatographed on a silica cartridge, eluting with a mixture (1/1) of dichloromethane and ethyl acetate. The fractions containing the expected product are combined and evaporated to dryness under reduced pressure, and the residue is concreted with a small amount of dichloromethane, pentane and ethyl ether to give 276 mg of 6-trimethylstannanyl-N-(pyridin-2-yl)imidazo[1,2-a]pyridine-2-carboxamide in the form of an off-white solid.

¹H NMR spectrum (DMSO-d6, δ in ppm): 0.37 (m, 9H), 7.19 (broad dd, J=5.0 and 8.0 Hz, 1H), 7.42 (broad d, J=9.5 Hz, 1H), 7.67 (d, J=9.5 Hz, 1H), 7.89 (m, 1H), 8.24 (d, J=8.0 Hz, 1H), 8.39 (broad d, J=5.0 Hz, 1H), 8.53 (m, 2H), 9.80 (s, 1H).

Mass spectrum (LC-MS-DAD-ELSD): m/z 403, [M+H]⁺

### Intermediate 19: Ethyl 6-(4,4,5,5-tetramethyl-1,3,2-dioxaborolan-2-yl)imidazo[1,2-a]pyridine-2-carboxylate hydrobromide (1:1)

To a solution of 4 g of 2-amino-5-(4,4,5,5-tetramethyl-1,3,2-dioxaborolan-2-yl)pyridine in 40 mL of 1,2-dimethoxyethane are added 4.26 g of ethyl 3-bromo-2-oxopropionate. The reaction mixture is stirred for 40 hours at 20° C. The precipitate is filtered off by suction, washed with a small amount of 1,2-dimethoxyethane and pentane and then taken up in 50 mL of ethanol and refluxed for 1 hour. The reaction mixture is concentrated to dryness under reduced pressure. The oil obtained is redissolved in ethyl ether and the solution is concentrated under reduced pressure. The solid is filtered off by suction and washed with a small amount of ethyl ether to give 3.78 g of ethyl 6-(4,4,5,5-tetramethyl-1,3,2-dioxaborolan-2-yl)imidazo[1,2-a]pyridine-2-carboxylate hydrobromide (1:1) in the form of a white solid.

¹H NMR spectrum (DMSO-d6, δ in ppm): 1.27-1.38 (m, 15H), 4.36 (q, J=7.3 Hz, 2H), 7.59 (d, J=9.3 Hz, 1H), 7.67 (d, J=9.3 Hz, 1H), 8.68 (s, 1H), 8.97 (s, 1H).

Mass spectrum (EI): m/z 316 [M]⁺, 244 [M−CO₂Et+H]⁺.

### Intermediate 20: 2-Ethoxycarbonylimidazo[1,2-a]pyridine-6-boronic acid

To a solution of 2.5 g of 2-amino-5-(4,4,5,5-tetramethyl-1,3,2-dioxaborolan-2-yl)pyridine in 50 mL of 1,2-dimethoxyethane are added 2.14 mL of ethyl 3-bromo-2-oxopropionate. The reaction mixture is stirred for 3.5 hours at 25° C., 50 mL of ethanol are then added and the mixture is refluxed for 16 hours. The reaction mixture is cooled and concentrated to dryness. The residue is suspended in 100 mL of water at 0° C. and treated by stirring vigorously with solid sodium carbonate until a pH of 8-9 is obtained. The precipitate is filtered off by suction and washed with 100 mL of water at 0° C. and then dissolved in 150 mL of methanol. The solution is dried over magnesium sulfate, filtered, concentrated and dried under vacuum to give 2.36 g of 2-ethoxycarbonylimidazo[1,2-a]pyridine-6-boronic acid in the form of a cream-coloured solid.

¹H NMR spectrum (DMSO-d6, δ in ppm): 8.82 (d, J=0.9, 1H), 8.58 (s, 1H), 8.35 (s, 2H) 7.61 (m, 2H), 4.33 (m, 2H), 1.32 (m, 3H)

Mass spectrum (APCI): m/z=235 [M+H]⁺.

The tables that follow illustrate the chemical structures (Table 1) and the spectroscopic characteristics (Table 2) of a number of examples of compounds according to the invention.

In these tables, -“2 HCl” means hydrochloride (1:2); “TFA” means trifluoroacetate (1:1); “-” means that the compound is in the form of the base.

TABLE 1
(I)

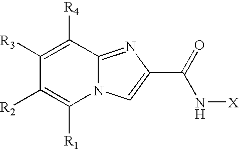

| Ex | R₁ | R₂ | R₃ | R₄ | X | salt |
|---|---|---|---|---|---|---|
| 1 | H | 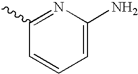 | H | H | 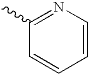 | 2 HCl |
| 2 | H | 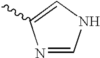 | H | H | 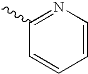 | — |
| 3 | H | 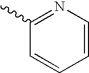 | H | H | 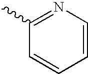 | — |
| 4 | H | 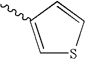 | H | H | 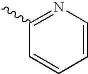 | — |
| 5 | H | 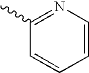 | H | H | 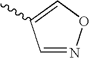 | — |
| 6 | H | 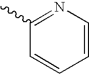 | H | H | 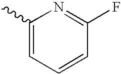 | — |
| 7 | H | 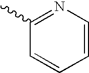 | H | H | 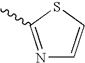 | — |
| 8 | H | 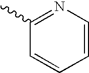 | H | H | 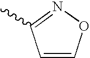 | — |
| 9 | H | 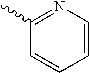 | H | H | 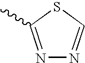 | — |
| 10 | H | 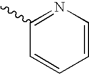 | H | H | 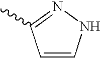 | — |
| 11 | H | 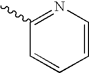 | H | H | 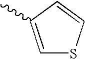 | — |
| 12 | H | 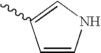 | H | H | 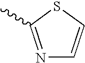 | — |
| 13 | H | 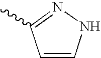 | H | H | 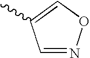 | — |
| 14 | H | 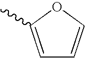 | H | H | 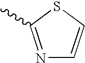 | — |
| 15 | H | 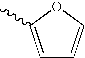 | H | H | 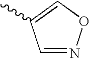 | — |
| 16 | H | 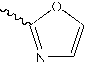 | H | H | 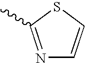 | — |
| 17 | H | 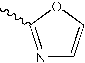 | H | H | 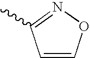 | — |
| 18 | H | 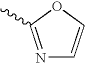 | H | H | 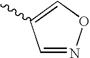 | — |
| 19 | H | 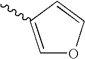 | H | H | 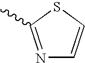 | — |
| 20 | H | 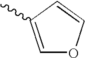 | H | H | 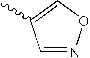 | — |
| 21 | H | 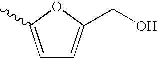 | H | H | 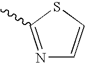 | — |
| 22 | H | 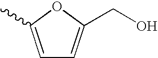 | H | H | 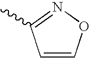 | — |
| 23 | H | 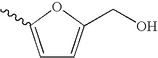 | H | H | 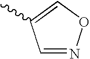 | — |
| 24 | H | 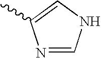 | H | H | 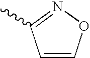 | — |
| 25 | H | 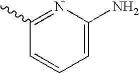 | H | H | 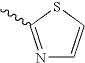 | — |
| 26 | H | 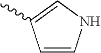 | H | H | 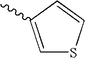 | — |
| 27 | H | 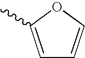 | H | H | 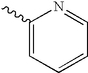 | — |
| 28 | H | 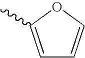 | H | H | 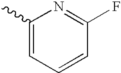 | — |
| 29 | H | 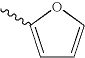 | H | H | 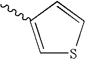 | — |
| 30 | H | 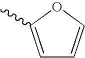 | H | H | 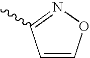 | — |
| 31 | H | 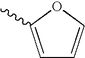 | H | H | 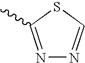 | — |
| 32 | H | 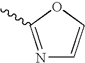 | H | H | 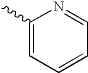 | — |
| 33 | H | 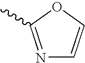 | H | H | 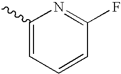 | — |
| 34 | H | 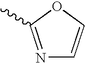 | H | H | 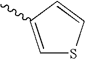 | — |
| 35 | H | 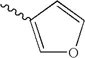 | H | H | 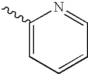 | — |
| 36 | H | 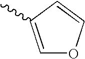 | H | H | 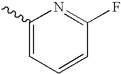 | — |
| 37 | H | 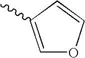 | H | H | 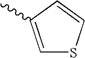 | — |
| 38 | H | 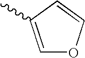 | H | H | 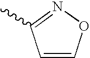 | — |
| 39 | H | 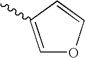 | H | H | 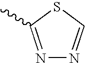 | — |
| 40 | H | 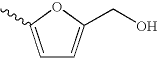 | H | H | 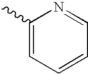 | — |
| 41 | H | 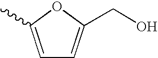 | H | H | 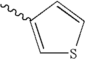 | — |
| 42 | H | 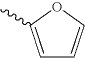 | H | H | 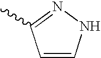 | — |
| 43 | H | 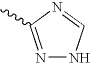 | H | H | 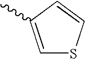 | — |
| 44 | H | 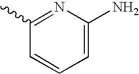 | H | H | 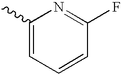 | 2 HCl |
| 45 | H | 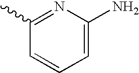 | H | H | 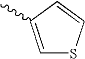 | 2 HCl |
| 46 | H | 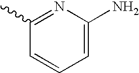 | H | H | 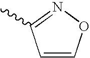 | 2 HCl |
| 47 | H | 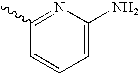 | H | H | 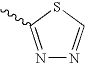 | 2 HCl |
| 48 | H | 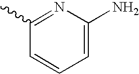 | H | H | 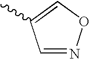 | 2 HCl |
| 49 | H | 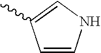 | H | H | 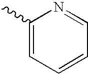 | TFA |
| 50 | H | 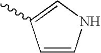 | H | H | 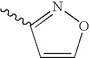 | — |
| 51 | H | 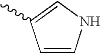 | H | H | 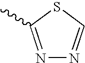 | — |
| 52 | H | 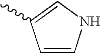 | H | H | 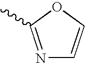 | — |
| 53 | H | 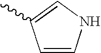 | H | H | 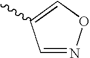 | — |
| 54 | H | 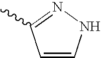 | H | H | 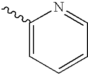 | — |
| 55 | H | 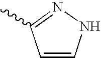 | H | H | 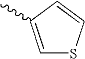 | TFA |
| 56 | H | 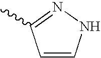 | H | H | 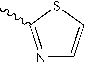 | TFA |
| 57 | H | 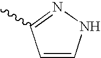 | H | H | 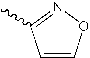 | — |
| 58 | H | 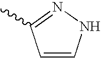 | H | H | 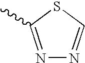 | TFA |
| 59 | H | 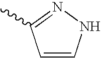 | H | H | 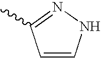 | — |
| 60 | H | 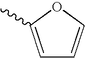 | H | H | 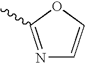 | TFA |
| 61 | H | 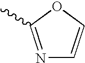 | H | H | 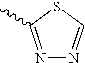 | — |
| 62 | H | 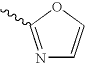 | H | H | 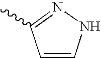 | — |
| 63 | H | 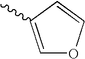 | H | H | 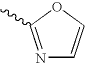 | TFA |
| 64 | H | 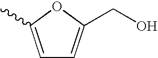 | H | H | 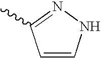 | — |
| 65 | H | 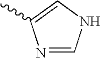 | H | H | 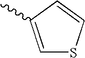 | — |
| 66 | H | 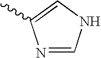 | H | H | 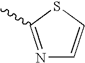 | — |
| 67 | H | 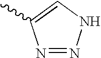 | H | H | 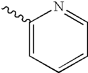 | — |
| 68 | H | 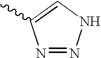 | H | H | 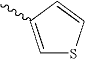 | — |

TABLE 2

| Ex | Characterizations |
|---|---|
| 1 | ¹H NMR spectrum (DMSO-d6, δ in ppm): 6.90 (broad m, 1H), from 7.30 to 7.39 (m, 2H), |
| | from 7.84 to 8.00 (m, 4H), 8.25 (d, J = 8.0 Hz, 1H), 8.42 (broad d, J = 5.0 Hz, 1H), 8.78 (s, |
| | 1H), 9.28 (broad s, 1H), 10.25 (broad m, 1H). |
| | Mass spectrum (LC-MS-DAD-ELSD): m/z 331 [M + H]⁺ |
| 2 | ¹H NMR spectrum (DMSO-d6, δ in ppm): 7.09 (m, 1H), from 7.64 to 7.95 (m, 5H), |
| | 8.26 (m, 1H), 8.39 (m, 1H), 8.65 (s, 1H), 9.01 (s, 1H), 9.79 (s, 1H), 12.3 (broad m, 1H). |
| | Mass spectrum (LC-MS-DAD-ELSD): m/z 305 [M + H]⁺, m/z 303 [M − H]⁻. |
| 3 | ¹H NMR spectrum (DMSO-d6, δ in ppm): 7.20 (ddd, J = 7.3, 4.9, 1.2 Hz, 1H), 7.43 (ddd, |
| | J = 7.3, 4.8, 1.2 Hz, 1H), 7.81 (dt, J = 9.6, 0.9 Hz, 1H), 7.89 (m, 1H), 7.96 (m, 1H), 8.02 (dt, |
| | J = 8.1, 1.2 Hz, 1H), 8.14 (dd, J = 9.6, 1.8 Hz, 1H), 8.26 (dt, J = 8.1, 1.2 Hz, 1H), 8.39 (ddd, |
| | J = 4.9, 2.0, 1.2 Hz, 1H), 8.71 (d, J = 0.9 Hz, 1H), 8.73 (m, 1H), 9.43 (dd, J = 1.8, 0.9 Hz, 1H), |
| | 9.83 (s, 1H). |
| | Mass spectrum (LC-MS-DAD-ELSD): m/z 316 [M + H]⁺ |
| 4 | ¹H NMR spectrum (DMSO-d6, δ in ppm): 7.19 (broad dd, J = 7.3, 4.9 Hz, 1H), 7.59 (dd, |
| | J = 5.9, 1.0 Hz, 1H), 7.72-7.79 (m, 2H), 7.82-7.92 (m, 2H), 8.01 (broad d, J = 3.4 Hz, 1H), |
| | 8.25 (broad d, J = 8.5 Hz, 1H), 8.39 (broad d, J = 4.9 Hz, 1H), 8.55 (s, 1H), 9.05 (broad s, |
| | 1H), 9.82 (s, 1H). |
| | Mass spectrum (LC-MS-DAD-ELSD): m/z 321 [M + H]⁺. |
| 5 | ¹H NMR spectrum (DMSO-d6, δ in ppm): 7.43 (ddd, J = 7.6, 4.8, 1.3 Hz, 1H), 7.75 (dt, |
| | J = 9.6, 1.0 Hz, 1H), 7.96 (td, J = 7.6, 1.9 Hz, 1H), 8.02 (dt, J = 7.6, 1.3 Hz, 1H), 8.12 (dd, |
| | J = 9.6, 1.9 Hz, 1H), 8.62 (d, J = 0.8 Hz, 1H), 8.71 (ddd, J = 4.8, 1.9, 1.3 Hz, 1H), 8.84 (s, |
| | 1H), 9.25 (s, 1H), 9.43 (dd, J = 1.9, 1.0 Hz, 1H), 10.94 (s, 1H). |
| | Mass spectrum (LC-MS-DAD-ELSD): m/z 306 [M + H]⁺, m/z 350 [M + HCO₂H − H]⁻. |
| 6 | ¹H NMR spectrum (DMSO-d6, δ in ppm): 6.95 (dd, J = 8.0, 2.5 Hz, 1H), 7.43 (m, 1H), |
| | 7.80 (d, J = 9.6 Hz, 1H), 7.88-8.16 (m, 5H), 8.68 (s, 1H), 8.70 (broad d, J = 5.0 Hz, 1H), |
| | 9.35 (broad s, 1H), 9.80 (broad s, 1H). |
| | Mass spectrum (LC-MS-DAD-ELSD): m/z 334 [M + H]⁺, m/z 378 [M + HCO₂H − H]⁻. |
| 7 | ¹H NMR spectrum (DMSO-d6, δ in ppm): 7.30 (d, J = 3.6 Hz, 1H), 7.43 (m, 1H), 7.55 (d, |
| | J = 3.6 Hz, 1H), 7.79 (d, J = 9.8 Hz, 1H), 7.97 (td, J = 7.7, 1.2 Hz, 1H), 8.03 (broad d, J = 7.7 Hz, |
| | 1H), 8.14 (dd, J = 9.8, 1.8 Hz, 1H), 8.72 (broad d, J = 4.9 Hz, 1H), 8.78 (s, 1H), |
| | 9.44 (broad s, 1H), 11.91 (broad s, 1H). |
| | Mass spectrum (LC-MS-DAD-ELSD): m/z 322 [M + H]⁺. |
| 8 | ¹H NMR spectrum (DMSO-d6, δ in ppm): 7.01 (d, J = 1.7 Hz, 1H), 7.43 (ddd, J = 7.5, 4.8, |
| | 1.3 Hz, 1H), 7.77 (dt, J = 9.6, 1.0 Hz, 1H), 7.96 (td, J = 7.5, 1.8 Hz, 1H), 8.03 (dt, J = 7.5, 1.3 Hz, |
| | 1H), 8.13 (dd, J = 9.6, 1.9 Hz, 1H), 8.70-8.73 (m, 2H), 8.84 (d, J = 1.7 Hz, 1H), |
| | 9.42 (dd, J = 1.9, 0.9 Hz, 1H), 10.95 (s, 1H). |
| | Mass spectrum (LC-MS-DAD-ELSD): m/z 306 [M + H]⁺. |
| 9 | ¹H NMR spectrum (DMSO-d6, δ in ppm): 7.41 (m, 1H), 7.77 (d, J = 9.5 Hz, 1H), |
| | 7.87-8.02 (m, 2H), 8.11 (dd, J = 9.5, 1.7 Hz, 1H), 8.70 (broad d, J = 4.9 Hz, 1H), 8.78 (s, 1H), |
| | 9.18 (s, 1H), 9.36 (broad s, 1H), 11.90 (broad m, 1H). |
| | Mass spectrum (LC-MS-DAD-ELSD): m/z 323 [M + H]⁺. |
| 10 | ¹H NMR spectrum (DMSO-d6, δ in ppm): 6.64 (broad s, 1H), 7.42 (ddd, J = 7.6, 4.8, 1.3 Hz, |
| | 1H), 7.68 (broad s, 1H), 7.77 (d, J = 9.5 Hz, 1H), 7.96 (td, J = 7.6, 1.9 Hz, 1H), |
| | 8.02 (broad d, J = 7.6 Hz, 1H), 8.11 (dd, J = 9.5, 1.8 Hz, 1H), 8.62 (s, 1H), 8.71 (broad d, J = 4.8 Hz, |
| | 1H), 9.42 (broad s, 1H), 9.90 (s, 1H), 12.46 (broad s, 1H). |
| | Mass spectrum (LC-MS-DAD-ELSD): m/z 305 [M + H]⁺. |
| 11 | ¹H NMR spectrum (DMSO-d6, δ in ppm): 7.39-7.49 (m, 2H), 7.52 (dd, J = 5.1, 1.4 Hz, |
| | 1H), 7.75 (d, J = 9.6 Hz, 1H), 7.80 (dd, J = 3.2, 1.4 Hz, 1H), 7.96 (td, J = 7.9, 1.9 Hz, 1H), |
| | 8.02 (broad d, J = 7.9 Hz, 1H), 8.11 (dd, J = 9.6, 1.9 Hz, 1H), 8.60 (s, 1H), 8.71 (broad d, |
| | J = 5.1 Hz, 1H), 9.42 (broad s, 1H), 10.83 (broad s, 1H). |
| | Mass spectrum (LC-MS-DAD-ELSD): m/z 321 [M + H]⁺. |
| 12 | ¹H NMR spectrum (DMSO-d6, δ in ppm): 6.49 (q, J = 2.5 Hz, 1H), 6.87 (dd, J = 2.5, 1.9 Hz, |
| | 1H), 7.29 (d, J = 3.6 Hz, 1H), 7.33 (dd, J = 2.5, 1.9 Hz, 1H), 7.54 (d, J = 3.6 Hz, 1H), |
| | 7.62 (broad d, J = 9.6 Hz, 1H), 7.70 (dd, J = 9.6, 1.9 Hz, 1H), 8.56 (s, 1H), 8.77 (broad s, 1H), |
| | 11.06 (broad s, 1H), 11.75 (broad s, 1 H). |
| | Mass spectrum (LC-MS-DAD-ELSD): m/z 310 [M + H]⁺, m/z 308 [M − H]⁻. |
| 13 | ¹H NMR spectrum (DMSO-d6, δ in ppm): (all the signals are broad) 6.71 (s, 1H), 7.66 (d, |
| | J = 9.5 Hz, 1H), 7.78 (s, 1H), 7.85 (d, J = 9.5 Hz, 1H), 8.49 (s, 1H), 8.82 (s, 1H), 9.02 (s, |
| | 1H), 9.16 (s, 1H), 10.60 (s, 1H), 12.86 (broad m, 1H). |
| | Mass spectrum (LC-MS-DAD-ELSD): m/z 295 [M + H]⁺, m/z 293 [M − H]⁻. |
| 14 | ¹H NMR spectrum (DMSO-d6, δ in ppm): 6.66 (dd, J = 3.4, 1.9 Hz, 1H), 7.07 (dd, J = 3.4, |
| | 0.9 Hz, 1H), 7.30 (d, J = 3.6 Hz, 1H), 7.55 (d, J = 3.6 Hz, 1H), 7.74 (broad d, J = 9.6 Hz, 1H), |
| | 7.78 (dd, J = 9.6, 1.7 Hz, 1H), 7.83 (dd, J = 1.9, 0.9 Hz, 1H), 8.72 (broad s, 1H), 9.00 (broad |
| | s, 1H), 11.86 (broad s, 1H). |
| | Mass spectrum (LC-MS-DAD-ELSD): m/z 311 [M + H]⁺. |
| 15 | ¹H NMR spectrum (DMSO-d6, δ in ppm): (all the signals are broad) 6.62 (m, 1H), 6.96 (d, |
| | J = 2.8 Hz, 1H), 7.66 (d, J = 9.6 Hz, 1H), 7.70 (d, J = 9.6 Hz, 1H), 7.76 (m, 1H), 8.51 (s, 1H), |
| | 8.82 (s, 1H), 8.94 (s, 1H), 9.15 (s, 1H), 10.59 (s, 1H). |
| | Mass spectrum (LC-MS-DAD-ELSD): m/z 295 [M + H]⁺, m/z 293 [M − H]⁻. |
| 16 | ¹H NMR spectrum (DMSO-d6, δ in ppm): 7.31 (d, J = 3.6 Hz, 1H), 7.46 (d, J = 0.9 Hz, 1H), |
| | 7.55 (d, J = 3.6 Hz, 1H), 7.83 (dt, J = 9.6, 0.9 Hz, 1H), 7.92 (dd, J = 9.6, 1.7 Hz, 1H), 8.32 (d, |
| | J = 0.9 Hz, 1H), 8.82 (d, J = 0.9 Hz, 1H), 9.40 (dd, J = 1.7, 0.9 Hz, 1H), 11.99 (broad s, 1H). |
| | Mass spectrum (LC-MS-DAD-ELSD): m/z 312 [M + H]⁺. |
| 17 | ¹H NMR spectrum (DMSO-d6, δ in ppm): 7.00 (d, J = 1.9 Hz, 1H), 7.46 (d, J = 0.9 Hz, 1H), |
| | 7.81 (dt, J = 9.6, 0.9 Hz, 1H), 7.92 (dd, J = 9.6, 1.9 Hz, 1H), 8.31 (d, J = 0.9 Hz, 1H), 8.74 (d, |
| | J = 0.9 Hz, 1H), 8.84 (d, J = 1.9 Hz, 1H), 9.38 (dd, J = 1.9, 0.9 Hz, 1H), 11.02 (s, 1H). |
| | Mass spectrum (LC-MS-DAD-ELSD): m/z 296 [M + H]⁺. |
| 18 | ¹H NMR spectrum (DMSO-d6, δ in ppm): 7.45 (d, J = 1.0 Hz, 1H), 7.79 (dt, J = 9.6, 0.9 Hz, |
| | 1H), 7.92 (dd, J = 9.6, 1.9 Hz, 1H), 8.31 (d, J = 1.0 Hz, 1H), 8.68 (d, J = 0.9 Hz, 1H), 8.83 (s, |
| | 1H), 9.26 (s, 1H), 9.38 (dd, J = 1.9, 0.9 Hz, 1H), 10.97 (s, 1H). |
| | Mass spectrum (LC-MS-DAD-ELSD): m/z 296 [M + H]⁺. |
| 19 | ¹H NMR spectrum (DMSO-d6, δ in ppm): 7.00 (dd, J = 1.8, 0.9 Hz, 1H), 7.29 (d, J = 3.6 Hz, |
| | 1H), 7.55 (d, J = 3.6 Hz, 1H), 7.72 (d, J = 1.4 Hz, 2H), 7.82 (t, J = 1.8 Hz, 1H), 8.31 (dd, |
| | J = 1.8, 0.9 Hz, 1H), 8.61 (s, 1H), 8.94 (t, J = 1.4 Hz, 1H), 11.87 (broad m, 1H). |
| | Mass spectrum (LC-MS-DAD-ELSD): m/z 311 [M + H]⁺. |
| 20 | ¹H NMR spectrum (DMSO-d6, δ in ppm): 6.97 (dd, J = 1.8, 0.9 Hz, 1H), 7.61-7.74 (m, |
| | 2H), 7.82 (t, J = 1.8 Hz, 1H), 8.29 (dd, J = 1.8, 0.9 Hz, 1H), 8.43 (s, 1H), 8.83 (s, 1H), |
| | 8.93 (t, J = 1.4 Hz, 1H), 9.24 (s, 1H), 10.89 (s, 1H). |
| | Mass spectrum (LC-MS-DAD-ELSD): m/z 295 [M + H]⁺, m/z 293 [M − H]⁻. |
| 21 | ¹H NMR spectrum (DMSO-d6, δ in ppm): 4.49 (d, J = 5.6 Hz, 2H), 5.31 (t, J = 5.6 Hz, 1H), |
| | 6.47 (d, J = 3.4 Hz, 1H), 6.99 (d, J = 3.4 Hz, 1H), 7.30 (d, J = 3.6 Hz, 1H), 7.55 (d, J = 3.6 Hz, |
| | 1H), 7.69-7.83 (m, 2H), 8.73 (s, 1H), 8.96 (broad s, 1H), 11.84 (broad m, 1H). |
| | Mass spectrum (LC-MS-DAD-ELSD): m/z 341 [M + H]⁺, m/z 339 [M − H]⁻. |
| 22 | ¹H NMR spectrum (DMSO-d6, δ in ppm): 4.49 (broad d, J = 5.6 Hz, 2H), 5.05 (broad m, |
| | 1H), 6.43 (d, J = 3.3 Hz, 1H), 6.91 (d, J = 3.3 Hz, 1H), 6.99 (broad s, 1H), 7.69 (m, 2H), |
| | 8.61 (s, 1H), 8.76 (broad s, 1H), 8.90 (broad s, 1H), 10.49 (broad m, 1H). |
| | Mass spectrum (LC-MS-DAD-ELSD): m/z 325 [M + H]⁺. |
| 23 | ¹H NMR spectrum (DMSO-d6, δ in ppm): 4.48 (d, J = 5.6 Hz, 2H), 5.30 (t, J = 5.6 Hz, 1H), |
| | 6.46 (d, J = 3.3 Hz, 1H), 6.97 (d, J = 3.3 Hz, 1H), 7.68 (dt, J = 9.5, 1.0 Hz, 1H), 7.74 (dd, |
| | J = 9.5, 1.7 Hz, 1H), 8.59 (d, J = 1.0 Hz, 1H), 8.83 (s, 1H), 8.96 (broad s, 1H), 9.24 (s, 1H), |
| | 10.89 (broad s, 1H). |
| | Mass spectrum (LC-MS-DAD-ELSD): m/z 325 [M + H]⁺, m/z 323 [M − H]⁻. |
| 24 | ¹H NMR spectrum (DMSO-d6, δ in ppm): 7.01 (d, J = 1.7 Hz, 1H), 7.66 (dt, J = 9.5, 0.9 Hz, |
| | 1H), 7.72 (dd, J = 1.9, 1.0 Hz, 1H), 7.78 (t, J = 1.0 Hz, 1H), 7.82 (dd, J = 9.5, 1.7 Hz, 1H), |
| | 8.64 (d, J = 0.9 Hz, 1H), 8.83 (d, J = 1.7 Hz, 1H), 8.99 (dd, J = 1.7, 0.9 Hz, 1H), 10.83 (broad |
| | s, 1H), 12.29 (broad m, 1H). |
| | Mass spectrum (LC-MS-DAD-ELSD): m/z 295 [M + H]⁺, m/z 293 [M − H]⁻. |
| 25 | ¹H NMR spectrum (DMSO-d6, δ in ppm): 7.01 (dd, J = 8.8, 1.0 Hz, 1H), 7.28 (dd, J = 7.4, |
| | 1.0 Hz, 1H), 7.33 (d, J = 3.6 Hz, 1H), 7.58 (d, J = 3.6 Hz, 1H), 7.92 (dt, J = 9.5, 1.0 Hz, 1H), |
| | 7.98 (dd, J = 8.8, 7.4 Hz, 1H), 8.06 (dd, J = 9.5, 1.7 Hz, 1H), 8.10 (very broad m, 2H), |
| | 8.87 (d, J = 1.0 Hz, 1H), 9.47 (dd, J = 2.0, 1.0 Hz, 1H), 10.67 (broad m, 1H). |
| | Mass spectrum (LC-MS-DAD-ELSD): m/z 337 [M + H]⁺, m/z 335 [M − H]⁻. |
| 26 | ¹H NMR spectrum (DMSO-d6, δ in ppm): 6.47 (m, 1H), 6.87 (m, 1H), 7.30 (m, 1H), |
| | 7.45 (dd, J = 5.1, 3.1 Hz, 1H), 7.50 (dd, J = 5.1, 1.4 Hz, 1H), 7.58 (d, J = 9.5 Hz, 1H), 7.66 (dd, |
| | J = 9.5, 1.7 Hz, 1H), 7.78 (dd, J = 3.1, 1.4 Hz, 1H), 8.38 (s, 1H), 8.76 (broad s, 1H), |
| | 10.71 (broad s, 1H), 11.04 (broad m, 1H) |
| | Mass spectrum (LC-MS-ES+/−): m/z 307 [M + H]⁻, m/z 309 [M + H]⁺ |
| 27 | ¹H NMR spectrum (DMSO-d6, δ in ppm): 6.66 (dd, J = 3.4, 1.9 Hz, 1H), 7.07 (d, J = 3.4 Hz, |
| | 1H), 7.19 (ddd, J = 7.4, 4.9, 0.9 Hz, 1H), 7.73-7.92 (m, 4H), 8.25 (d, J = 8.4 Hz, 1H), |
| | 8.38 (ddd, J = 4.9, 1.9, 0.9 Hz, 1H), 8.65 (s, 1H), 8.99 (broad s, 1H), 9.79 (broad s, 1H) |
| | Mass spectrum (LC-MS-ES+/−): m/z 305 [M + H]⁺ |
| 28 | ¹H NMR spectrum (DMSO-d6, δ in ppm): 6.66 (dd, J = 3.4, 1.9 Hz, 1H), 6.94 (ddd, J = 7.8, |
| | 2.5, 0.8 Hz, 1H), 7.07 (dd, J = 3.4, 0.8 Hz, 1H), 7.75 (d, J = 9.5 Hz, 1H), 7.80 (dd, J = 9.5, 1.7 Hz, |
| | 1H), 7.83 (dd, J = 1.9, 0.8 Hz, 1H), 8.06 (q, J = 8.1 Hz, 1H), 8.14 (ddd, J = 8.1, 2.5, 0.8 Hz, |
| | 1H), 8.67 (s, 1H), 8.98 (broad s, 1H), 9.89 (broad s, 1H) |
| | Mass spectrum (LC-MS-ES+/−): m/z 323 [M + H]⁺ |
| 29 | ¹H NMR spectrum (DMSO-d6, δ in ppm): 3.65 (dd, J = 3.5, 1.9 Hz, 1H), 7.04 (dd, J = 3.5, |
| | 0.8 Hz, 1H), 7.46 (dd, J = 5.1, 3.3 Hz, 1H), 7.51 (dd, J = 5.1, 1.4 Hz, 1H), 7.69 (d, J = 9.6 Hz, |
| | 1H), 7.75 (dd, J = 9.6, 1.7 Hz, 1H), 7.79 (dd, J = 3.3, 1.4 Hz, 1H), 7.82 (dd, J = 1.9, 0.8 Hz, |
| | 1H), 8.55 (s, 1H), 8.99 (broad s, 1H), 10.78 (broad s, 1H) |
| | Mass spectrum (LC-MS-ES+/−): m/z 310 [M + H]⁺ |
| 30 | ¹H NMR spectrum (DMSO-d6, δ in ppm): 6.66 (dd, J = 3.4, 1.9 Hz, 1H), 7.01 (d, J = 1.7 Hz, |
| | 1H), 7.06 (dd, J = 3.4, 0.8 Hz, 1H), 7.72 (d, J = 9.5 Hz, 1H), 7.77 (dd, J = 9.5, 1.7 Hz, 1H), |
| | 7.83 (dd, J = 1.9, 0.8 Hz, 1H), 8.64 (s, 1H), 8.83 (d, J = 1.7 Hz, 1H), 8.99 (broad s, 1H), |
| | 10.90 (broad s, 1H) |
| | Mass spectrum (LC-MS-ES+/−): m/z 295 [M + H]⁺ |
| 31 | ¹H NMR spectrum (DMSO-d6, δ in ppm): 6.66 (dd, J = 3.4, 1.9 Hz, 1H), 7.08 (d, J = 3.4 Hz |
| | 1H), 7.69-7.86 (m, 3H), 8.77 (s, 1H), 9.01 (broad s, 1H), 9.24 (broad s, 1H), |
| | 12.47-12.80 (broad m, 1H) |
| | Mass spectrum (LC-MS-ES+/−): m/z 310 [M + H]⁻, m/z 312 [M + H]⁺ |
| 32 | ¹H NMR spectrum (DMSO-d6, δ in ppm): 7.17-7.23 (m, 1H), 7.44-7.47 (m, 1H), |
| | 7.82-7.96 (m, 3H), 8.24 (d, J = 8.3 Hz, 1H), 8.30-8.32 (m, 1H), 8.37-8.41 (m, 1H), 8.75 (s, 1H), |
| | 9.37 (broad s, 1H), 9.83 (broad s, 1H) |
| | Mass spectrum (LC-MS-ES+/−): m/z 306 [M + H]⁺ |
| 33 | ¹H NMR spectrum (DMSO-d6, δ in ppm): 6.95 (dd, J = 7.9, 2.3 Hz, 1H), 7.44-7.47 (m, 1H), |
| | 7.84 (d, J = 9.5 Hz, 1H), 7.93 (dd, J = 9.5, 1.7 Hz, 1H), 8.01-8.17 (m, 2H), 8.3-8.33 (m, 1H), |
| | 8.77 (s, 1H), 9.37 (broad s, 1H), 9.95 (s, 1H) |
| | Mass spectrum (LC-MS-ES+/−): m/z 324 [M + H]⁺ |
| 34 | ¹H NMR spectrum (DMSO-d6, δ in ppm): 7.44-7.53 (m, 3H), 7.76-7.82 (m, 2H), 7.9 (dd, |
| | J = 9.5, 1.7 Hz, 1H), 8.30-8.31 (m, 1H), 8.65 (s, 1H), 9.38 (broad s, 1H), 10.85 (broad s, |
| | 1H) |
| | Mass spectrum (LC-MS-ES+/−): m/z 311 [M + H]⁺ |
| 35 | ¹H NMR spectrum (DMSO-d6, δ in ppm): 6.98 (dd, J = 1.9, 0.9 Hz, 1H), 7.19 (ddd, J = 7.3, |
| | 4.9, 1.1 Hz, 1H), 7.73-7.74 (m, 2H), 7.82 (t, J = 1.9 Hz, 1H), 7.89 (m, 1H), 8.25 (dt, J = 8.4, |
| | 0.9 Hz, 1H), 8.3 (broad s, 1H), 8.39 (ddd, J = 4.9, 1.9, 0.9 Hz, 1H), 8.52 (s, 1H), 8.92 (broad |
| | s, 1H), 9.8 (s, 1H) |
| | Mass spectrum (LC-MS-ES+/−): m/z 305 [M + H]⁺ |
| 36 | ¹H NMR spectrum (DMSO-d6, δ in ppm): 6.94 (ddd, J = 8.1, 2.6, 0.9 Hz, 1H), 6.98 (dd, |
| | J = 1.9, 0.9 Hz, 1H), 7.74 (m, 2H), 7.82 (t, J = 1.9 Hz, 1H), 8.06 (q, J = 8.1 Hz, 1H), 8.15 (ddd, |
| | J = 8.1, 2.6, 0.9 Hz, 1H), 8.3 (broad s, 1H), 8.55 (s, 1H), 8.92 (broad s, 1H), 9.91 (broad s, |
| | 1H) |
| | Mass spectrum (LC-MS-ES+/−): m/z 323 [M + H]⁺ |
| 37 | ¹H NMR spectrum (DMSO-d6, δ in ppm): 6.98 (dd, J = 1.8, 0.9 Hz, 1H), 7.46 (dd, J = 5.3, |
| | 3.3 Hz, 1H), 7.5 (dd, J = 5.3, 1.4 Hz, 1H), 7.68 (m, 2H), 7.78 (dd, J = 3.3, 1.4 Hz, 1H), |
| | 7.82 (t, J = 1.8 Hz, 1H), 8.29 (broad s, 1H), 8.42 (s, 1H), 8.93 (broad s, 1H), 10.84 (broad s, 1H) |
| | Mass spectrum (LC-MS-ES+/−): m/z 310 [M + H]⁺ |
| 38 | ¹H NMR spectrum (DMSO-d6, δ in ppm): 6.99 (dd, J = 1.9, 0.9 Hz, 1H), 7.01 (d, J = 1.9 Hz, |
| | 1H), 7.7 (m, 2H), 7.82 (t, J = 1.9 Hz, 1H), 8.30 (broad s, 1H), 8.52 (s, 1H), 8.83 (d, J = 1.9 Hz, |
| | 1H), 8.93 (broad s, 1H), 10.9 (broad s, 1H) |
| | Mass spectrum (LC-MS-ES+/−): m/z 295 [M + H]⁺ |
| 39 | ¹H NMR spectrum (DMSO-d6, δ in ppm): 7.00 (dd, J = 1.9, 0.9 Hz, 1H), 7.73 (m, 2H), |
| | 7.82 (t, J = 1.9 Hz, 1H), 8.31 (broad s, 1H), 8.67 (s, 1H), 8.95 (broad s, 1H), 9.23 (s, 1H), |
| | 12.58 (broad m, 1H) |
| | Mass spectrum (LC-MS-ES+/−): m/z 310 [M + H]⁻, m/z 312 [M + H]⁺ |
| 40 | ¹H NMR spectrum (DMSO-d6, δ in ppm): 4.49 (d, J = 5.6 Hz, 2H), 5.30 (t, J = 5.6 Hz, 1H), |
| | 6.47 (d, J = 3.3 Hz, 1H), 6.99 (d, J = 3.3 Hz, 1H), 7.14-7.24 (m, 1H), 7.76 (s, 2H), |
| | 7.83-7.95 (m, 1H), 8.23 (d, J = 8.2 Hz, 1H), 8.34-8.43 (m, 1H), 8.66 (s, 1H), 8.95 (broad s, 1H), |
| | 9.79 (broad s, 1H) |
| | Mass spectrum (LC-MS-ES+/−): m/z 335 [M + H]⁺ |
| 41 | ¹H NMR spectrum (DMSO-d6, δ in ppm): 4.48 (d, J = 5.6 Hz, 2H), 5.30 (t, J = 5.6 Hz, 1H), |
| | 6.46 (d, J = 3.5 Hz, 1H), 6.97 (d, J = 3.5 Hz, 1H), 7.46 (dd, J = 5.6, 3.2 Hz, 1H), 7.50 (dd, |
| | J = 5.6, 1.5 Hz, 1H), 7.65-7.75 (m, 2H), 7.78 (dd, J = 3.2, 1.5 Hz, 1H), 8.58 (s, 1H), |
| | 8.95 (broad s, 1H), 10.78 (broad s, 1H) |
| | Mass spectrum (LC-MS-ES+/−): m/z 340 [M + H]⁺ |
| 42 | ¹H NMR spectrum (DMSO-d6, δ in ppm): 6.61 (broad m, 1H), 6.66 (dd, J = 3.5, 1.2 Hz, |
| | 1H), 7.05 (broad d, J = 3.5 Hz, 1H), 7.66 (broad m, 1H), 7.69-7.80 (m, 2H), 7.82 (broad d, |
| | J = 1.2 Hz, 1H), 8.56 (s, 1H), 8.98 (broad s, 1H), 9.89 (broad m, 1H), 12.47 (broad m, 1H) |
| | Mass spectrum (LC-MS-ES+/−): m/z 292 [M + H]⁻, m/z 294 [M + H]⁺ |
| 43 | ¹H NMR spectrum (DMSO-d6, δ in ppm): 7.46 (dd, J = 5.2, 3.2 Hz.1H), 7.52 (dd, J = 5.2, |
| | 1.3 Hz, 1H), 7.75 (d, J = 9.5 Hz, 1H), 7.79 (dd, J = 3.2, 1.3 Hz, 1H), 7.97 (dd, J = 9.5, 1.6 Hz, |
| | 1H), 8.65 (s, 1H), 8.69 (broad m, 1H), 9.31 (broad s, 1H), 10.81 (broad s, 1H), |
| | 14.27 (broad m, 1H) |
| | Mass spectrum (LC-MS-ES+/−): m/z 309 [M + H]⁻, m/z 311 [M + H]⁺ |
| 44 | ¹H NMR spectrum (DMSO-d6, δ in ppm): 6.82-6.89 (m, 1H), 6.93-6.99 (m, 1H), 7.21 (d, |
| | J = 7.1 Hz, 1H), 7.50 (broad m, 2H), 7.82-7.98 (m, 3H), 8.02-8.18 (m, 2H), 8.78 (s, 1H), |
| | 9.26 (s, 1H), 10.13 (broad s, 1H) |
| | Mass spectrum (LC-MS-ES+/−): m/z 349 [M + H]⁺ |
| 45 | ¹H NMR spectrum (DMSO-d6, δ in ppm): 6.98 (d, J = 8.6 Hz, 1H), 7.25 (d, J = 7.3 Hz, 1H), |
| | 7.45-7.54 (m, 2H), 7.76 (broad m, 2H), 7.77-7.90 (m, 2H), 7.90-8.04 (m, 2H), 8.66 (s, 1H), |
| | 9.35 (broad s, 1H), 11.01 (broad s, 1H) |
| | Mass spectrum (LC-MS-ES+/−): m/z 336 [M + H]⁺ |
| 46 | ¹H NMR spectrum (DMSO-d6, δ in ppm): 6.98 (d, J = 8.7 Hz, 1H), 7.02 (dd, J = 1.6 Hz, 1H), |
| | 7.26 (d, J = 7.3 Hz, 1H), 7.88 (d, J = 9.5 Hz, 1H), 7.93-8.01 (m, 2H), 8.14 (broad m, 3 H), |
| | 8.77 (s, 1H), 8.86 (d, J = 1.6 Hz, 1H), 9.38 (broad s, 1H), 11.23 (s, 1H) |
| | Mass spectrum (LC-MS-ES+/−): m/z 321 [M + H]⁺, m/z = 365 [M + HCO₂H − H]⁻ |
| 47 | ¹H NMR spectrum (DMSO-d6, δ in ppm): 7.01 (broad d, J = 8.6 Hz, 1H), 7.28 (broad d, |
| | J = 7.6 Hz, 1H), 7.84-8.04 (m, 3H), 8.20 (broad m, 3H), 8.90 (s, 1H), 9.25 (s, 1H), |
| | 9.44 (broad s, 1H) |
| | Mass spectrum (LC-MS-ES+/−): m/z 336 [M + H]⁻, m/z 338 [M + H]⁺ |
| 48 | ¹H NMR spectrum (DMSO-d6, δ in ppm): 6.98 (d, J = 8.8 Hz, 1H), 7.24 (d, J = 7.3 Hz, 1H), |
| | 7.84 (d, J = 9.5 Hz, 1H), 7.88-8.02 (m, 2H), 8.08 (broad m, 3 H), 8.63 (s, 1H), 8.84 (s, 1H), |
| | 9.26 (s, 1H), 9.32 (s, 1H), 11.05 (s, 1H) (broad signals) |
| | Mass spectrum (LC-MS-ES+/−): m/z 321 [M + H]⁺, m/z = 365 [M + HCO₂H − H]⁻ |
| 49 | ¹H NMR spectrum (DMSO-d6, δ in ppm): 6.51 (m, 1H), 6.89 (m, 1H), 7.23 (ddd, J = 7.4, |
| | 5.0, 1.1 Hz, 1H), 7.37 (m, 1H), 7.69 (d, J = 9.5 Hz, 1H), 7.86 (dd, J = 9.5, 1.7 Hz, 1H), |
| | 7.93 (ddd, J = 8.4, 7.4, 2.0 Hz, 1H), 8.24 (dt, J = 8.4, 1.1 Hz, 1H), 8.41 (ddd, J = 5.0, 2.0, 1.1 Hz, |
| | 1H), 8.65 (s, 1H), 8.87 (broad s, 1H), 10.42 (broad m, 1H), 11.12 (broad m, 1H) |
| | Mass spectrum (LC-MS-ES+/−): m/z 302 [M + H]⁻, m/z 304 [M + H]⁺ |
| 50 | ¹H NMR spectrum (DMSO-d6, δ in ppm): 6.47 (m, 1H), 6.87 (m, 1H), 7.01 (d, J = 1.6 Hz, |
| | 1H), 7.31 (m, 1H), 7.61 (d, J = 9.5 Hz, 1H), 7.69 (dd, J = 9.5, 1.7 Hz, 1H), 8.48 (s, 1H), |
| | 8.76 (broad s, 1H), 8.83 (d, J = 1.6 Hz, 1H), 10.81 (broad s, 1H), 11.05 (broad m, 1H) |
| | Mass spectrum (LC-MS-ES+/−): m/z 292 [M + H]⁻, m/z 294 [M + H]⁺ |
| 51 | ¹H NMR spectrum (DMSO-d6, δ in ppm): 6.49 (m, 1H), 6.87 (m, 1H), 7.33 (m, 1H), |
| | 7.63 (d, J = 9.5 Hz, 1H), 7.72 (dd, J = 9.5, 1.7 Hz, 1H), 8.62 (s, 1H), 8.78 (broad s, 1H), 9.22 (s, |
| | 1H), 11.06 (broad m, 1H), 12.49 (very broad m, 1H) |
| | Mass spectrum (LC-MS-ES+/−): m/z 309 [M + H]⁻, m/z 311 [M + H]⁺ |
| 52 | ¹H NMR spectrum (DMSO-d6, δ in ppm): 6.47 (m, 1H), 6.87 (m, 1H), 7.17 (s, 1H), |
| | 7.32 (m, 1H), 7.59 (d, J = 9.5 Hz, 1H), 7.69 (dd, J = 9.5, 1.2 Hz, 1H), 7.95 (s, 1H), 8.46 (s, 1H), |
| | 8.75 (s, 1H), 10.95-11.16 (m, 2H) (broad signals) |
| | Mass spectrum (LC-MS-ES+/−): m/z 292 [M + H]⁻, m/z 294 [M + H]⁺ |
| 53 | ¹H NMR spectrum (DMSO-d6, δ in ppm): 6.46 (m, 1H), 6.87 (m, 1H), 7.30 (m, 1H), |
| | 7.58 (d, J = 9.7 Hz, 1H), 7.68 (dd, J = 9.7, 1.7 Hz, 1H), 8.40 (s, 1H), 8.77 (broad s, 1H), 8.83 (s, |
| | 1H), 9.23 (s, 1H), 10.83 (broad s, 1H), 11.04 (broad m, 1H) |
| | Mass spectrum (LC-MS-ES+/−): m/z 292 [M + H]⁻, m/z 294 [M + H]⁺ |
| 54 | ¹H NMR spectrum (DMSO-d6, δ in ppm): 6.77 (d, J = 2.3 Hz, 1H), 7.19 (m, 1H), 7.75 (d, |
| | J = 9.5 Hz, 1H), 7.79-7.99 (m, 3H), 8.25 (d, J = 8.2 Hz, 1H), 8.39 (m, 1H), 8.61 (s, 1H), |
| | 9.09 (broad s, 1H), 9.81 (broad s, 1H), 13.06 (broad m, 1H) |
| | Mass spectrum (LC-MS-ES+/−): m/z 303 [M + H]⁻, m/z 305 [M + H]⁺ |
| 55 | ¹H NMR spectrum (DMSO-d6, δ in ppm): 6.77 (d, J = 2.3 Hz, 1H), 7.46 (dd, J = 5.2, 3.2 Hz, |
| | 1H), 7.50 (dd, J = 5.2, 1.4 Hz, 1H), 7.70 (d, J = 9.5 Hz, 1H), 7.79 (dd, J = 3.2, 1.4 Hz, 1H), |
| | 7.84 (d, J = 2.3 Hz, 1H), 7.90 (dd, J = 9.5, 1.7 Hz, 1H), 8.52 (s, 1H), 9.10 (broad s, 1H), |
| | 10.81 (s, 1H), 13.03 (broad m, 1H) |
| | Mass spectrum (LC-MS-ES+/−): m/z 308 [M + H]⁻, m/z 310 [M + H]⁺ |
| 56 | ¹H NMR spectrum (DMSO-d6, δ in ppm): 6.81 (d, J = 2.3 Hz, 1H), 7.31 (d, J = 3.7 Hz, 1H), |
| | 7.57 (d, J = 3.7 Hz, 1H), 7.78 (d, J = 9.6 Hz, 1H), 7.86 (d, J = 2.3 Hz, 1H), 8.02 (dd, J = 9.6, 1.7 Hz, |
| | 1H), 8.74 (s, 1H), 9.18 (broad s, 1H), 11.55-12.61 (broad m, 1H) |
| | Mass spectrum (LC-MS-ES+/−): m/z 309 [M + H]⁻, m/z 311 [M + H]⁺ |
| 57 | ¹H NMR spectrum (DMSO-d6, δ in ppm): 6.77 (broad s, 1H), 7.01 (d, J = 1.7 Hz, 1H), |
| | 7.71 (d, J = 9.6 Hz, 1H), 7.86 (broad s, 1H), 7.90 (broad d, J = 9.6 Hz, 1H), 8.61 (s, 1H), 8.83 (d, |
| | J = 1.7 Hz, 1H), 9.09 (broad s, 1H), 10.89 (s, 1H), 13.05 (broad s, 1H) |
| | Mass spectrum (LC-MS-ES+/−): m/z 293 [M + H]⁻, m/z 295 [M + H]⁺ |
| 58 | ¹H NMR spectrum (DMSO-d6, δ in ppm): 6.79 (broad d, J = 2.2 Hz, 1H), 7.74 (d, J = 9.5 Hz, |
| | 1H), 7.85 (broad s, 1H), 7.91 (broad d, J = 9.5 Hz.1H), 8.74 (s, 1H), 9.11 (broad s, 1H), |
| | 9.23 (s, 1H), 12.36-12.78 (broad m, 1H), 12.97-13.16 (broad s, 1H) |
| | Mass spectrum (LC-MS-ES+/−): m/z 310 [M + H]⁻, m/z 312 [M + H]⁺ |
| 59 | ¹H NMR spectrum (DMSO-d6, δ in ppm): 6.61 (s, 1H), 6.76 (d, J = 1.9 Hz, 1H), 7.70 (m, |
| | 2H), 7.80-7.94 (m, 2H), 8.53 (s, 1H), 9.08 (s, 1H), 9.79-10.01 (broad m, 1H), |
| | 12.38-12.54 (broad m, 1H), 13.04 (s, 1H), (broad signals) |
| | Mass spectrum (LC-MS-ES+/−): m/z 292 [M + H]⁻, m/z 294 [M + H]⁺ |
| 60 | ¹H NMR spectrum (DMSO-d6, δ in ppm): 6.67 (dd, J = 3.4, 1.7 Hz, 1H), 7.08 (dd, J = 3.4, |
| | 0.6 Hz, 1H), 7.20 (d, J = 0.8 Hz, 1H), 7.73 (d, J = 9.6 Hz, 1H), 7.79-7.87 (m, 2H), 7.95 (d, |
| | J = 0.8 Hz, 1H), 8.65 (s, 1H), 9.01 (broad s, 1H), 11.09 (very broad m, 1H) |
| | Mass spectrum (LC-MS-ES+/−): m/z 295 [M + H]⁺ |
| 61 | ¹H NMR spectrum (DMSO-d6, δ in ppm): 7.46 (m, 1H), 7.84 (d, J = 9.5 Hz, 1H), |
| | 7.90-7.97 (dd, J = 9.5, 1.7 Hz, 1H), 8.32 (m, 1H), 8.87 (s, 1H), 9.24 (s, 1H), 9.41 (broad s, 1H) |
| | Mass spectrum (LC-MS-ES+/−): m/z 311 [M + H]⁻, m/z 313 [M + H]⁺ |
| 62 | ¹H NMR spectrum (DMSO-d6, δ in ppm): 6.49-6.68 (broad m, 1H), 7.43 (s, 1H), |
| | 7.58-7.74 (broad m, 1H), 7.75-7.85 (d, J = 9.5 Hz, 1H), 7.85-7.96 (dd, J = 9.5, 1.7 Hz, 1H), |
| | 8.30 (s, 1H), 8.67 (s, 1H), 9.38 (broad s, 1H), 9.79-10.11 (broad m, 1H), 12.36-12.56 (broad m, |
| | 1H) |
| | Mass spectrum (LC-MS-ES+/−): m/z 293 [M + H]⁻, m/z 295 [M + H]⁺ |
| 63 | ¹H NMR spectrum (DMSO-d6, δ in ppm): 7.02 (dd, J = 1.9, 0.9 Hz, 1H), 7.29 (broad s, |
| | 1H), 7.80 (d, J = 9.5 Hz, 1H), 7.86 (t, J = 1.9 Hz, 1H), 7.94 (broad s, 1H), 8.00 (dd, J = 9.5, 1.1 Hz, |
| | 1H), 8.37 (broad s, 1H), 8.64 (s, 1H), 9.09 (broad s, 1H) |
| | Mass spectrum (LC-MS-ES+/−): m/z 295 [M + H]⁺ |
| 64 | ¹H NMR spectrum (DMSO-d6, δ in ppm): 4.48 (d, J = 5.7 Hz, 2H), 5.29 (t, J = 5.7 Hz, 1H), |
| | 6.46 (d, J = 3.3 Hz, 1H), 6.58-6.66 (broad m, 1H), 6.97 (d, J = 3.3 Hz, 1H), 7.56-7.80 (m, |
| | 3H), 8.58 (s, 1H), 8.94 (broad s, 1H), 9.77-9.95 (broad m, 1H), 12.33-12.57 (broad m, 1H) |
| | Mass spectrum (LC-MS-ES+/−): m/z 322 [M + H]⁻, m/z 324 [M + H]⁺ |
| 65 | ¹H NMR spectrum (DMSO-d6, δ in ppm): 7.41-7.53 (m, 2H), 7.63 (d, J = 9.6 Hz, 1H), |
| | 7.70 (broad s, 1H), 7.74-7.84 (m, 3H), 8.53 (s, 1H), 9.00 (broad s, 1H), 10.74 (broad s, 1H), |
| | 12.27 (broad m, 1H) |
| | Mass spectrum (LC-MS-ES+/−): m/z 308 [M + H]⁻, m/z 310 [M + H]⁺ |
| 66 | ¹H NMR spectrum (DMSO-d6, δ in ppm): 6.62-6.72 (m, 1H), 7.22 (d, J = 3.8 Hz, 1H), |
| | 7.55 (d, J = 9.4 Hz, 1H), 7.60-7.69 (m, 2H), 7.76 (s, 1H), 8.27 (broad s, 1H), 8.92 (broad s, 1H), |
| | 12.27 (broad m, 1H) |
| | Mass spectrum (LC-MS-ES+/−): m/z 309 [M + H]⁻, m/z 311 [M + H]⁺ |
| 67 | ¹H NMR spectrum (DMSO-d6, δ in ppm): 7.19 (ddd, J = 7.4, 4.9, 1.0 Hz, 1H), 7.81 (d, |
| | J = 9.6 Hz, 1H), 7.85-7.94 (m, 2H), 8.25 (dt, J = 8.3, 1.0 Hz, 1H), 8.39 (ddd, J = 4.9, 2.0, 1.0 Hz, |
| | 1H), 8.45 (broad m, 1H), 8.67 (s, 1H), 9.20 (broad s, 1H), 9.82 (broad s, 1H), |
| | 15.29 (broad m, 1H) |
| | Mass spectrum (LC-MS-ES+/−): m/z 306 [M + H]⁺ |
| 68 | ¹H NMR spectrum (DMSO-d6, δ in ppm): 7.46 (dd, J = 5.1, 3.2 Hz, 1H), 7.51 (dd, J = 5.1, |
| | 1.5 Hz, 1H), 7.75 (d, J = 9.5 Hz, 1H), 7.79 (dd, J = 3.2, 1.5 Hz, 1H), 7.86 (dd, J = 9.5, 1.7 Hz, |
| | 1H), 8.42 (broad m, 1H), 8.55 (s, 1H), 9.20 (broad s, 1H), 10.80 (broad s, 1H), |
| | 15.26 (broad m, 1H) |
| | Mass spectrum (LC-MS-ES+/−): m/z 309 [M + H]⁻, m/z 311 [M + H]^{+x} |

The compounds according to the invention underwent pharmacological tests to determine their modulatory effect on NOT.

Evaluation of the in Vitro Activity on N2A Cells

The activity of the compounds according to the invention was evaluated on a cell line (N2A) endogenously expressing the murine Nurr1 receptor and stably transfected with the NOT binding response element (NBRE) coupled to the luciferase reporter gene. The EC₅₀ values are between 0.01 and 1000 nM. The tests were performed according to the procedure described hereinbelow.

The cell line Neuro-2A is obtained from a standard commercial source (ATCC). The clone Neuro-2A was obtained from a spontaneous tumour originating from a strain of albino mice A by R. J Klebe et al. This line Neuro-2A is then stably transfected with 8NBRE-luciferase. The N2A-8NBRE cells are cultured to the point of confluence in 75 cm² culture flasks containing DMEM supplemented with 10% foetal calf serum, 4.5 g/L of glucose and 0.4 mg/ml of geneticin. After culturing for one week, the cells are recovered with 0.25% trypsin for 30 seconds and then resuspended in DMEM without phenol red, containing 4.5 g/L of glucose and 10% Hyclone defatted serum, and placed in white, transparent-based 96-well plates. The cells are deposited at a rate of 60 000 per well in 75 μL for 24 hours before adding the products. The products are applied in 25 μL and incubated for a further 24 hours. On the day of measurement, an equivalent volume (100 μL) of Steadylite is added to each well, and the wells are then left for 30 minutes to obtain complete lysis of the cells and maximum production of the signal. The plates are then measured in a microplate luminescence counter, after having been sealed with an adhesive film. The products are prepared in the form of a 10⁻² M stock solution, and then diluted in 100% of DMSO. Each concentration of product is prediluted in culture medium before incubation with the cells thus containing 0.625% final of DMSO.

For example, compounds 1, 2, 7, 10, 22, 36, 51, 56 and 58 gave an EC₅₀ value of 16 nM, 1.5 nM, 0.8 nM, 21 nM, 2 nM, 0.4 nM, 1.5 nM, 1 nM and 5.3 nM, respectively.

It is thus seen that the compounds according to the invention have a modulatory effect on NOT.

The compounds according to the invention may thus be used for the preparation of medicaments for their therapeutic application in the treatment or prevention of diseases involving the NOT receptors.

Thus, according to another of its aspects, a subject of the invention is medicaments comprising a compound of formula (I), or an addition salt thereof with a pharmaceutically acceptable acid.

These medicaments find their therapeutic use especially in the treatment and prevention of neurodegenerative diseases, for instance Parkinson's disease, Alzheimer's disease, tauopathies (e.g. progressive supranuclear palsy, frontotemporal dementia, corticobasal degeneration, Pick's disease); cerebral trauma, for instance ischaemia and cranial trauma and epilepsy; psychiatric diseases, for instance schizophrenia, depression, substance dependency, and attention-deficit hyperactivity disorder; inflammatory diseases of the central nervous system, for instance multiple sclerosis, encephalitis, myelitis and encephalomyelitis and other inflammatory diseases, for instance vascular pathologies, atherosclerosis, joint inflammations, arthrosis, rheumatoid arthritis; osteoarthritis, Crohn's disease, ulcerative colitis; allergic inflammatory diseases such as asthma, autoimmune diseases, for instance type 1 diabetes, lupus, scleroderma, Guillain-Barré syndrome, Addison's disease and other immune-mediated diseases; osteoporosis; cancers.

Thus, one subject of the present invention is directed towards a compound of formula (I) as defined previously, for the treatment of the abovementioned diseases, complaints and disorders.

According to another of its aspects, the present invention relates to the use of a compound of formula (I) as defined previously, for the preparation of a medicament for treating or preventing one of the diseases, complaints or disorders mentioned above.

These compounds may also be used as a treatment combined with grafts and/or transplantations of stem cells.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt of the said compound, and also at least one pharmaceutically acceptable excipient.

The said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the salt thereof, may be administered in unit administration form, as a mixture with standard pharmaceutical excipients, to man and animals for the prophylaxis or treatment of the above complaints or diseases.

The appropriate unit forms of administration include oral forms such as tablets, soft or hard gel capsules, powders, granules and oral solutions or suspensions, sublingual, buccal, intratracheal, intraocular, intranasal or inhalation administration forms, topical, transdermal, subcutaneous, intramuscular or intravenous administration forms, rectal administration forms and implants. For topical application, the compounds according to the invention may be used in creams, gels, ointments or lotions.

By way of example, a unit administration form of a compound according to the invention in tablet form may comprise the following components:

| | |
|---|---|
| Compound according to the invention | 50.0 mg |
| Mannitol | 223.75 mg |
| Croscarmellose sodium | 6.0 mg |
| Corn starch | 15.0 mg |
| Hydroxypropylmethylcellulose | 2.25 mg |
| Magnesium stearate | 3.0 mg |

There may be particular cases in which higher or lower dosages are appropriate; such dosages are not outside the context of the invention. According to the usual practice, the dosage that is appropriate for each patient is determined by the doctor according to the mode of administration and the weight and response of the said patient.

According to another of its aspects, the present invention also relates to a method for treating the pathologies indicated above, which comprises the administration, to a patient, of an effective dose of a compound according to the invention, or a pharmaceutically acceptable salt thereof.

It is understood that all the subjects of the invention defined above, especially the medicament, pharmaceutical composition and treatment method, also apply more particularly to the subgroups of compounds previously defined.

What is claimed is:

1. The compound corresponding to formula (I): 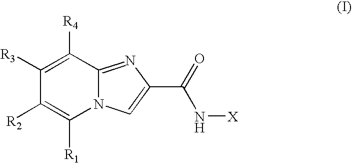
wherein:
X represents a heterocyclic group optionally substituted with one or more groups chosen, independently of each other, from the following groups or atoms: halogen, (C₁-C₆)alkoxy, (C₁-C₆)alkyl, NRaRb, cyano, oxido, and COOR₈, the alkyl and alkoxy groups possibly being substituted with one or more halogen atoms;
R₁ represents a hydrogen atom, a halogen atom, a group (C₁-C₆)alkoxy, a group (C₁-C₆)alkyl, amino or NRaRb; the alkyl and alkoxy groups possibly being substituted with one or more halogen atoms, a hydroxyl or amino group, or a group (C₁-C₆)alkoxy;
R₂ represents a heterocyclic group, this group possibly being substituted with one or more groups chosen, independently of each other, from the following groups or atoms: hydroxyl, (C₁-C₆)alkyl, (C₁-C₆)alkoxy, halogen, cyano, NRaRb, —CO—R₅, —CO—NR₆R₇, —CO—O—R₈, and —NR₉—CO—R₁₀, the groups (C₁-C₆)alkyl and (C₁-C₆)alkoxy being optionally substituted with one or more halogen atoms or hydroxyl, NRaRb or oxido groups;
R₃ represents a hydrogen atom, a group (C₁-C₆)alkyl, a group (C₁-C₆)alkoxy or a halogen atom;
R₄ represents a hydrogen atom, a group (C₁-C₄)alkyl, a group (C₁-C₄)alkoxy or a fluorine atom;
R₅ represents a hydrogen atom, a phenyl group or a group (C₁-C₆)alkyl;
R₆ and R₇, which may be identical or different, represent a hydrogen atom or a group (C₁-C₆)alkyl, or form, with the nitrogen atom that bears them, a 4- to 7-membered ring optionally including another heteroatom chosen from N, O and S;
R₈ represents a group (C₁-C₆)alkyl;
R₉ and R₁₀, which may be identical or different, represent a hydrogen atom or a group (C₁-C₆)alkyl;
Ra and Rb represent, independently of each other, a hydrogen atom, a group (C₁-C₆)alkyl or form, with the nitrogen atom that bears them, a 4- to 7-membered ring optionally including another heteroatom chosen from N, O and S;
or an acid addition salt thereof.

2. The compound of formula (I) according to claim 1, wherein:
X represents a heterocyclic group optionally substituted with one or more groups chosen, independently of each other, from halogen atoms;
or an acid addition salt thereof.

3. The compound of formula (I) according to claim 1, wherein:
R₁, R₃ and R₄ represent a hydrogen atom;
or an acid addition salt thereof.

4. The compound of formula (I) according to claim 1, wherein:
R₂ represents a heterocyclic group, this group optionally being substituted with one or more groups chosen, independently of each other, from the following groups or atoms: (C₁-C₆)alkyl, and NRaRb, the group(s) (C₁-C₆)alkyl being optionally substituted with one or more halogen atoms or hydroxyl groups; and
Ra and Rb represent, independently of each other, a hydrogen atom or a group (C₁-C₆)alkyl;
or an acid addition salt thereof.

5. The compound of formula (I) according to claim 1, wherein:
X represents a pyridine, isoxazole, thiazole, thiadiazole, pyrazole, thiophene or oxazole group, these groups being optionally substituted with a fluorine atom;
R₂ represents pyridine, thiophene, imidazole, pyrrole, furan, oxazole, triazole or pyrazole group, these groups being optionally substituted with an NH₂ or hydroxymethyl group; and
R₁, R₃ and R₄ represent a hydrogen atom;
or an acid addition salt thereof.

6. The compound of formula (I) according to claim 1, selected from the group consisting of:
6-(6-Aminopyridin-2-yl)-N-(pyridin-2-yl)imidazo[1,2-a]pyridine-2-carboxamide and its hydrochloride (1:2);
6-(1H-Imidazol-4-yl)-N-(pyridin-2-yl)imidazo[1,2-a]pyridine-2-carboxamide;
N,6-Di(pyridin-2-yl)imidazo[1,2-a]pyridine-2-carboxamide;
N-(Pyridin-2-yl)-6-(thiophen-3-yl)imidazo[1,2-a]pyridine-2-carboxamide;
N-(Isoxazol-4-yl)-6-(pyridin-2-yl)imidazo[1,2-a]pyridine-2-carboxamide;
N-(6-Fluoropyridin-2-yl)-6-(pyridin-2-yl)imidazo[1,2-a]pyridine-2-carboxamide;
6-(Pyridin-2-yl-N-(thiazol-2-yl)imidazo[1,2-a]pyridine-2-carboxamide;
N-(Isoxazol-3-yl)-6-(pyridin-2-yl)imidazo[1,2-a]pyridine-2-carboxamide;
6-(Pyridin-2-yl-N-(1,3,4-thiadiazol-2-yl)imidazo[1,2-a]pyridine-2-carboxamide;
N-(1H-Pyrazol-3-yl)-6-(pyridin-2-yl)imidazo[1,2-a]pyridine-2-carboxamide;
6-(Pyridin-2-yl-N-(thiophen-3-yl)imidazo[1,2-a]pyridine-2-carboxamide;
6-(1H-Pyrrol-3-yl)-N-(thiazol-2-yl)imidazo[1,2-a]pyridine-2-carboxamide;
N-(Isoxazol-4-yl)-6-(1H-pyrazol-3-yl)imidazo[1,2-a]pyridine-2-carboxamide;
6-(Furan-2-yl)-N-(thiazol-2-yl)imidazo[1,2-a]pyridine-2-carboxamide;
6-(Furan-2-yl)-N-(isoxazol-4-yl)imidazo[1,2-a]pyridine-2-carboxamide;
6-(Oxazol-2-yl)-N-(thiazol-2-yl)imidazo[1,2-a]pyridine-2-carboxamide;
N-(Isoxazol-3-yl)-6-(oxazol-2-yl)imidazo[1,2-a]pyridine-2-carboxamide;
N-(Isoxazol-4-yl)-6-(oxazol-2-yl)imidazo[1,2-a]pyridine-2-carboxamide;
6-(Furan-3-yl)-N-(thiazol-2-yl)imidazo[1,2-a]pyridine-2-carboxamide;
6-(Furan-3-yl)-N-(isoxazol-4-yl)imidazo[1,2-a]pyridine-2-carboxamide;
6-[5-(Hydroxymethyl)furan-2-yl]-N-(thiazol-2-yl)imidazo[1,2-a]pyridine-2-carboxamide;
6-[5-(Hydroxymethyl)furan-2-yl]-N-(isoxazol-3-yl)imidazo[1,2-a]pyridine-2-carboxamide;
6-[5-(Hydroxymethyl)furan-2-yl]-N-(isoxazol-4-yl)imidazo[1,2-a]pyridine-2-carboxamide;
6-(1H-Imidazol-4-yl)-N-(isoxazol-3-yl)imidazo[1,2-a]pyridine-2-carboxamide;
6-(6-Aminopyridin-2-yl)-N-(thiazol-2-yl)imidazo[1,2-a]pyridine-2-carboxamide;
6-(1H-Pyrrol-3-yl)-N-(thiophen-3-yl)imidazo[1,2-a]pyridine-2-carboxamide;
6-(Furan-2-yl)-N-(pyridin-2-yl)imidazo[1,2-a]pyridine-2-carboxamide;
N-(6-Fluoropyridin-2-yl)-6-(furan-2-yl)imidazo[1,2-a]pyridine-2-carboxamide;
6-(Furan-2-yl)-N-(thiophen-3-yl)imidazo[1,2-a]pyridine-2-carboxamide;
6-(Furan-2-yl)-N-(isoxazol-3-yl)imidazo[1,2-a]pyridine-2-carboxamide;
6-(Furan-2-yl)-N-(1,3,4-thiadiazol-2-yl)imidazo[1,2-a]pyridine-2-carboxamide;
6-(Oxazol-2-yl)-N-(pyridin-2-yl)imidazo[1,2-a]pyridine-2-carboxamide;
N-(6-Fluoropyridin-2-yl)-6-(1,3-oxazol-2-yl)imidazo[1,2-a]pyridine-2-carboxamide;
6-(Oxazol-2-yl)-N-(thiophen-3-yl)imidazo[1,2-a]pyridine-2-carboxamide;
6-(Furan-3-yl)-N-(pyridin-2-yl)imidazo[1,2-a]pyridine-2-carboxamide;
N-(6-Fluoropyridin-2-yl)-6-(furan-3-yl)imidazo[1,2-a]pyridine-2-carboxamide;
6-(Furan-3-yl)-N-(thiophen-3-yl)imidazo[1,2-a]pyridine-2-carboxamide;
6-(Furan-3-yl)-N-(isoxazol-3-yl)imidazo[1,2-a]pyridine-2-carboxamide;
6-(Furan-3-yl)-N-(1,3,4-thiadiazol-2-yl)imidazo[1,2-a]pyridine-2-carboxamide;
6-[5-(Hydroxymethyl)furan-2-yl]-N-(pyridin-2-yl)imidazo[1,2-a]pyridine-2-carboxamide;
6-[5-(Hydroxymethyl)furan-2-yl]-N-(thiophen-3-yl)imidazo[1,2-a]pyridine-2-carboxamide;
6-(Furan-2-yl)-N-(1H-pyrazol-3-yl)imidazo[1,2-a]pyridine-2-carboxamide;
N-(Thiophen-3-yl)-6-(1H-1,2,4-triazol-3-yl)imidazo[1,2-a]pyridine-2-carboxamide;
6-(6-Aminopyridin-2-yl)-N-(6-fluoropyridin-2-yl)imidazo[1,2-a]pyridine-2-carboxamide and its hydrochloride (1:2);
6-(6-Aminopyridin-2-yl)-N-(thiophen-3-yl)imidazo[1,2-a]pyridine-2-carboxamide and its hydrochloride (1:2);
6-(6-Aminopyridin-2-yl)-N-(isoxazol-3-yl)imidazo[1,2-a]pyridine-2-carboxamide and its hydrochloride (1:2);
6-(6-Aminopyridin-2-yl)-N-(1,3,4-thiadiazol-2-yl)imidazo[1,2-a]pyridine-2-carboxamide and its hydrochloride (1:2);
6-(6-Aminopyridin-2-yl)-N-(isoxazol-4-yl)imidazo[1,2-a]pyridine-2-carboxamide and its hydrochloride (1:2);
N-(Pyridin-2-yl)-6-(1H-pyrrol-3-yl)imidazo[1,2-a]pyridine-2-carboxamide and its trifluoroacetate (1:1);
N-(Isoxazol-3-yl)-6-(1H-pyrrol-3-yl)imidazo[1,2-a]pyridine-2-carboxamide;
6-(1H-Pyrrol-3-yl)-N-(1,3,4-thiadiazol-2-yl)imidazo[1,2-a]pyridine-2-carboxamide;
N-(Oxazol-2-yl)-6-(1H-pyrrol-3-yl)imidazo[1,2-a]pyridine-2-carboxamide;
N-(Isoxazol-4-yl)-6-(1H-pyrrol-3-yl)imidazo[1,2-a]pyridine-2-carboxamide;
6-(1H-Pyrazol-3-yl)-N-(pyridin-2-yl)imidazo[1,2-a]pyridine-2-carboxamide;
6-(1H-Pyrazol-3-yl)-N-(thiophen-3-yl)imidazo[1,2-a]pyridine-2-carboxamide and its trifluoroacetate (1:1);
6-(1H-Pyrazol-3-yl)-N-(1,3-thiazol-2-yl)imidazo[1,2-a]pyridine-2-carboxamide and its trifluoroacetate (1:1);
N-(Isoxazol-3-yl)-6-(1H-pyrazol-3-yl)imidazo[1,2-a]pyridine-2-carboxamide;
6-(1H-Pyrazol-3-yl)-N-(1,3,4-thiadiazol-2-yl)imidazo[1,2-a]pyridine-2-carboxamide and it trifluoroacetate (1:1);
N,6-Di(1H-pyrazol-3-yl)imidazo[1,2-a]pyridine-2-carboxamide;
6-(Furan-2-yl)-N-(1,3-oxazol-2-yl)imidazo[1,2-a]pyridine-2-carboxamide and its trifluoroacetate (1:1);
6-(Oxazol-2-yl)-N-(1,3,4-thiadiazol-2-yl)imidazo[1,2-a]pyridine-2-carboxamide;
6-(Oxazol-2-yl)-N-(1H-pyrazol-3-yl)imidazo[1,2-a]pyridine-2-carboxamide;
6-(Furan-3-yl)-N-(1,3-oxazol-2-yl)imidazo[1,2-a]pyridine-2-carboxamide and its trifluoroacetate (1:1);
6-[5-(Hydroxymethyl)furan-2-yl]-N-(1H-pyrazol-3-yl)imidazo[1,2-a]pyridine-2-carboxamide;
6-(1H-Imidazol-4-yl)-N-(thiophen-3-yl)imidazo[1,2-a]pyridine-2-carboxamide;
6-(1H-Imidazol-4-yl)-N-(1,3-thiazol-2-yl)imidazo[1,2-a]pyridine-2-carboxamide;
N-(Pyridin-2-yl)-6-(1H-1,2,3-triazol-4-yl)imidazo[1,2-a]pyridine-2-carboxamide; and
N-(Thiophen-3-yl)-6-(1H-1,2,3-triazol-4-yl)imidazo[1,2-a]pyridine-2-carboxamide;
or an acid addition salt thereof.

7. A pharmaceutical composition comprising a compound of formula (I) according to claim 1, or a pharmaceutically acceptable salt thereof, and also at least one pharmaceutically acceptable excipient.

8. A pharmaceutical composition comprising a compound of formula (I) according to claim 6, or a pharmaceutically acceptable salt thereof, and also at least one pharmaceutically acceptable excipient.

9. A method for treating or preventing neurodegenerative diseases comprising administering to a patient an effective amount of a compound of formula (I) according to claim 1 or a pharmaceutically acceptable salt thereof.

10. A method for treating or preventing cerebral trauma or epilepsy comprising administering to a patient an effective amount of a compound of formula (I) according to claim 1 or a pharmaceutically acceptable salt thereof.

11. A method for treating or preventing psychiatric diseases comprising administering to a patient an effective amount of a compound of formula (I) according to claim 1 or a pharmaceutically acceptable salt thereof.

12. A method for treating or preventing inflammatory diseases comprising administering to a patient an effective amount of a compound of formula (I) according to claim 1 or a pharmaceutically acceptable salt thereof.

13. A method for treating or preventing osteoporosis comprising administering to a patient an effective amount of a compound of formula (I) according to claim 1 or a pharmaceutically acceptable salt thereof.

14. A method for treating or preventing cancers comprising administering to a patient an effective amount of a compound of formula (I) according to claim 1 or a pharmaceutically acceptable salt thereof.

15. A method for treating or preventing Parkinson's disease, Alzheimer's disease, tauopathies or multiple sclerosis comprising administering to a patient an effective amount of a compound of formula (I) according to claim 1 or a pharmaceutically acceptable salt thereof.

16. A method for treating or preventing schizophrenia, depression, substance dependency or attention-deficit hyperactivity disorder comprising administering to a patient an effective amount of a compound of formula (I) according to claim 1 or a pharmaceutically acceptable salt thereof.

17. A compound selected from the group consisting of:
6-(6-{[(1,1-Dimethylethoxy)carbonyl]amino}pyridin-2-yl)imidazo[1,2-a]pyridine-2-carboxylic acid;
6-(Pyridin-2-yl)imidazo[1,2-a]pyridine-2-carboxylic acid;
6-(1-Triphenylmethyl-1H-imidazol-4-yl)imidazo[1,2-a]pyridine-2-carboxylic acid;
6-(2-{[(1,1-Dimethylethoxy)carbonyl]amino}thiazol-4-yl)imidazo[1,2-a]pyridine-2-carboxylic acid;
6-(1H-Pyrrol-3-yl)imidazo[1,2-a]pyridine-2-carboxylic acid;
6-(1H-Pyrazol-3-yl)imidazo[1,2-a]pyridine-2-carboxylic acid;
6-(1H-Pyrazol-4-yl)imidazo[1,2-a]pyridine-2-carboxylic acid;
6-(Furan-2-yl)imidazo[1,2-a]pyridine-2-carboxylic acid;
6-(Furan-3-yl)imidazo[1,2-a]pyridine-2-carboxylic acid;
6-[5-(Hydroxymethyl)furan-2-yl]imidazo[1,2-a]pyridine-2-carboxylic acid;
6-(Thiophen-3-yl)imidazo[1,2-a]pyridine-2-carboxylic acid;
6-(Oxazol-2-yl)imidazo[1,2-a]pyridine-2-carboxylic acid;
6-(1H-1,2,4-Triazol-3-yl)imidazo[1,2-a]pyridine-2-carboxylic acid;
6-(1H-1,2,3-Triazol-4-yl)imidazo[1,2-a]pyridine-2-carboxylic acid;
6-Iodo-N-(isoxazol-4-yl)imidazo[1,2-a]pyridine-2-carboxamide;
6-Iodo-N-(thiazol-2-yl)imidazo[1,2-a]pyridine-2-carboxamide;
6-Iodo-N-(pyridin-2-yl)imidazo[1,2-a]pyridine-2-carboxamide;
6-Trimethylstannyl-N-(pyridin-2-yl)imidazo[1,2-a]pyridine-2-carboxamide;
Ethyl 6-(4,4,5,5-tetramethyl-1,3,2-dioxaborolan-2-yl)imidazo[1,2-a]pyridine-2-carboxylate hydrobromide (1:1); and
2-Ethoxycarbonylimidazo[1,2-a]pyridine-6-boronic acid.
